(19) United States
(12) Patent Application Publication
(10) Pub. No.: US 2007/0254843 A1
(43) Pub. Date: Nov. 1, 2007

(54) Methods for treating bone associated diseases by the use of methionine aminopeptidase-2 inhibitors

(75) Inventors: Gerhard Hannig, Revere, MA (US); William F. Westlin, Boxborough, MA (US)

Correspondence Address: LAHIVE & COCKFIELD, LLP, ONE POST OFFICE SQUARE, BOSTON, MA, 02109-2127, US

(73) Assignee: Praecis Pharmaceuticals Incorporated, Waltham, MA (US)

(21) Appl. No.: 11/787,846

(22) Filed: Apr. 18, 2007

Related U.S. Application Data
(60) Provisional application No. 60/792,827, filed on Apr. 18, 2006.

(51) Int. Cl. A61K 38/16; A61K 31/336

(52) U.S. Cl. 514/12; 514/475; 514/13; 514/14; 514/15; 514/16; 514/17; 514/18; 514/19

(57) ABSTRACT

The instant invention provides methods and compositions for treating a subject suffering from bone associated diseases, such as osteoporosis.

### RELATED APPLICATION

This application claims priority to U.S. Provisional Application No. 60/792,827, filed Apr. 18, 2006, the entire contents of which are incorporated herein by this reference.

### BACKGROUND OF THE INVENTION

Bone erosion is mediated by osteoclasts (OC), highly specialized multinucleated cells which are derived from hematopoietic precursors. Unregulated bone resorption by OC, however, may lead to the development of bone associated diseases in which the amount of bone in a subject is decreased or the structural integrity of the bone is impaired. Bone associated diseases include, but are not limited to, osteoporosis, Paget's Disease, Gorham's Disease, multiple myeloma, bone metastasis of cancer, periodontal disease, renal osteodystrophy, Hajdu-Cheney Syndrome (acro-osteolysis), Idiopathic Multicentric Osteolysis, Multicentric Osteolysis with nephropathy, Torg Osteolysis Syndrome (multicentric osteolysis), Neurogenic osteolysis, Joseph and Shinz Disease (Idiopathic Phalangeal Acro-osteolysis), Winchester Syndrome, Lupus, and Kummell's Disease.

There is no known cure for bone associated diseases. Current treatment goals include the reduction of pain and discomfort, the prevention of deformities and loss of joint function, and the suppression of inflammation. The three general classes of drugs commonly used in the treatment of bone associated disease include non-steroidal anti-inflammatory agents (NSAIDs), which act to reduce acute inflammation; corticosteroids, which have both anti-inflammatory and immunoregulatory activity; and disease modifying anti-rheumatic drugs (DMARDs). Only DMARDs have been shown to improve disease outcome by inhibiting molecular mechanisms such as TNF-α, receptor activator of nuclear factor-kB (RANK), and RANKL, which are involved in the pathology of bone associated diseases. A significant number of patients, however, have not responded to any of these treatments, have become refractory to available agents, or had treatment interrupted due to intolerable side effects.

Accordingly, there is still a need for effective treatments for these diseases, e.g., methods for reducing the differentiation and bone resorption of osteoclasts within a subject.

### SUMMARY OF THE INVENTION

The present invention provides methods of treating a bone associated disease in a subject. The methods include administering to the subject a therapeutically effective amount of a methionine aminopeptidase 2 inhibitor, thereby treating a bone associated disease, e.g., osteoporosis, in a subject. The present invention is based, at least in part, on the discovery that Met-AP2 inhibitors potently inhibit the differentiation and bone resorption of osteoclasts.

In one aspect, the invention provides a method of treating a bone associated disease in a subject, e.g., a human, by administering to the subject a therapeutically effective amount of a methionine aminopeptidase 2 inhibitor, thereby treating a bone associated disease in a subject. In one embodiment, the bone associated disease is selected from the group consisting of osteoporosis, Paget's Disease, Gorham's Disease, multiple myeloma, bone metastasis of cancer, periodontal disease, renal osteodystrophy, Hajdu-Cheney Syndrome (acro-osteolysis), Idiopathic Multicentric Osteolysis, Multicentric Osteolysis with nephropathy, Torg Osteolysis Syndrome (multicentric osteolysis), Neurogenic osteolysis, Joseph and Shinz Disease (Idiopathic Phalangeal Acro-osteolysis), Winchester Syndrome, Lupus, and Kummell's Disease.

In one embodiment, the methionine aminopeptidase 2 inhibitor is a compound of Formula I, 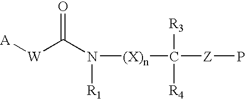
wherein A is a Met-AP2 inhibitory core; W is O or NR₂; R₁ and R₂ are each, independently, hydrogen or alkyl; X is alkylene or substituted alkylene; n is 0 or 1; R₃ and R₄ are each, independently, hydrogen, substituted or unsubstituted alkyl, substituted or unsubstituted aryl or substituted or unsubstituted heteroaryl; or R₃ and R₄, together with the carbon atom to which they are attached, form a carbocyclic or heterocyclic group; or R₃ and R₄ together form an alkylene group; Z is —C(O)— or alkylene-C(O)—; and
P is a peptide comprising from 1 to about 100 amino acid residues attached at its amino terminus to Z or a group OR₅ or N(R₆)R₇, wherein
R₅, R₆ and R₇ are each, independently, hydrogen, alkyl, substituted alkyl, azacycloalkyl or substituted azacycloalkyl; or R₆ and R₇, together with the nitrogen atom to which they are attached, form a substituted or unsubstituted heterocyclic ring structure; or
Z is —O—, —NR₈—, alkylene-O— or alkylene-NR₈—, where R₈ is hydrogen or alkyl; and
P is hydrogen, alkyl or a peptide consisting of from 1 to about 100 amino acid residues attached at its carboxy terminus to Z.

In another embodiment, the methionine aminopeptidase 2 inhibitor is a compound of Formula XV, 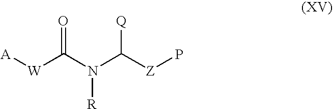
wherein A is a MetAP-2 inhibitory core; W is O or NR; each R is, independently, hydrogen or alkyl; Z is —C(O)— or -alkylene-C(O)—; P is NHR, OR or a peptide consisting of one to about one hundred amino acid residues connected at the N-terminus to Z; Q is hydrogen, linear, branched or cyclic alkyl or aryl, provided that when P is —OR, Q is not hydrogen; or Z is -alkylene-O— or -alkylene-N(R)—; P is hydrogen or a peptide consisting of from one to about one hundred amino acid residues connected to Z at the carboxyl terminus; Q is hydrogen, linear, branched or cyclic alkyl or aryl, provided that when P is hydrogen, Q is not hydrogen; and pharmaceutically acceptable salts thereof.

In yet another embodiment, the methionine aminopeptidase 2 inhibitor is a compound of the formula 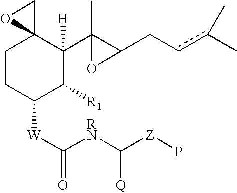
wherein W is O or NR; each R is, independently hydrogen or a C₁-C₄-alkyl; Q is hydrogen; linear, branched or cyclic C₁-C₆-alkyl; or aryl; R¹ is hydroxy, C₁-C₄-alkoxy or halogen; Z is —C(O)— or C₁-C₄-alkylene; P is NHR, OR, or a peptide comprising 1 to 100 amino acid residues attached to Z at the N-terminus; or Z is alkylene-O or alkylene-NR; and P is hydrogen or peptide comprising 1 to 100 amino acid residues attached to Z at the C-terminus; or a pharmaceutically acceptable salt thereof; provided that when P is hydrogen, NHR or OR, Q is not hydrogen.

In a further embodiment, the methionine aminopeptidase 2 inhibitor is a compound comprising the structure 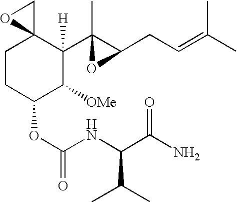
or a pharmaceutically acceptable salt thereof.

In one embodiment, the methionine aminopeptidase 2 inhibitor is administered at a dosage range of about 0.1 and 30 mg/kg or about 0.1 and 10 mg/kg.

In another embodiment, the methionine aminopeptidase 2 inhibitor may be administered to the subject in a sustained-release formulation, e.g., a sustained-release formulation which provides sustained delivery of the methionine aminopeptidase 2 inhibitor to a subject for at least one, two, three, four or five weeks after the formulation is administered to the subject.

In another aspect, the present invention provides a method of treating osteoporosis in a subject, e.g., a human. The method includes administering to the subject a therapeutically effective amount of a methionine aminopeptidase 2 inhibitor comprising the structure (1-Carbamoyl-2-methyl-propyl)-carbamic acid-(3R,4S,5S,6R)-5-methoxy-4-[(2R,3R)-2-methyl-3-(3-methyl-but-2-enyl)-oxiranyl]-1-oxa-spiro[2.5]oct-6-yl ester, or a pharmaceutically acceptable salt thereof, thereby treating osteoporosis in a subject.

Other features and advantages of the invention will be apparent from the following detailed description and claims.

### BRIEF DESCRIPTION OF THE DRAWINGS

FIG. 1 is a graph depicting the inhibition of osteoclast differentiation and bone resorption in vitro by the MetAP-2 inhibitor used in the present studies. FIG. 1A depicts OC differentiation, cultured in the presence of either M-CSF, RANKL, or the MetAP-2 inhibitor used in the present studies. FIG. 1B depicts an ELISA for CTX-I after primary human OC precursors were combined with human bone particles and cultured in the presence of either M-CSF, RANKL, vehicle E-64, or the MetAP-2 inhibitor used in the present studies.

FIG. 2 is a graph illustrating that the MetAP-2 inhibitor used in the present studies has potent anti-inflammatory activity in the rat model of PG-PS-induced arthritis. Rats were dosed with vehicle, dexamethasone, or MetAP-2 inhibitor, and the volumes of the two hind paws were measured and averaged.

FIG. 3 is a graph demonstrating that the inhibition of MetAP-2 in vivo by a MetAP-2 inhibitor is correlated with the suppression of chronic arthritis. The amount of MetAP-2 inhibited in wbc lysates was determined by the MetAP-2 pharmacodynamic assay.

FIG. 4 is a graph illustrating that the MetAP-2 inhibitor used in the present studies inhibits cartilage erosion in the rat PG-PS arthritis model. The amount of COMP, a mediator of chondrocyte attachment, in serum was measured by ELISA.

FIG. 5 is a graph portraying the inhibition of bone resorption in the rat PG-PS arthritis model by the MetAP-2 inhibitor used in the present studies. The amount of CTX-I, a marker for bone resorption, in urine was measured by ELISA.

FIG. 6 contains images of hind paws and illustrates that the MetAP-2 inhibitor used in the present studies preserves the joint architecture as evidenced by three-dimensional rendered micro-CT analysis.

FIG. 7 depicts two graphs showing the markers of bone destruction from the animal model of osteoporosis treated with the MetAP-2 inhibitors as described herein.

### DETAILED DESCRIPTION OF THE INVENTION

The present invention provides methods of treating a bone associated disease in a subject. The methods include administering to the subject a therapeutically effective amount of a methionine aminopeptidase 2 inhibitor, thereby treating a bone associated disease, e.g., osteoporosis, in a subject. The present invention is based, at least in part, on the discovery that Met-AP2 inhibitors potently inhibit the differentiation and bone resorption of osteoclasts.

As used herein, the term “bone associated disease” is intended to include any disease, disorder or condition in which the amount of bone in a subject is modulated, e.g., decreased or increased, and/or the structural integrity of the bone is impaired. This term includes diseases, disorders, or conditions in which bone erosion mediated by bone resorption by osteoclasts occurs. Bone associated diseases include, but are not limited to, osteoporosis, Paget's Disease, Gorham's Disease, multiple myeloma, bone metastasis of cancer, periodontal disease, renal osteodystrophy, Hajdu-Cheney Syndrome (acro-osteolysis), Idiopathic Multicentric Osteolysis, Multicentric Osteolysis with nephropathy, Torg Osteolysis Syndrome (multicentric osteolysis), Neurogenic osteolysis, Joseph and Shinz Disease (Idiopathic Phalangeal Acro-osteolysis), Winchester Syndrome, Lupus, and Kummell's Disease. In one embodiment, this term does not include diseases such as cancer or inflammatory diseases, e.g., rheumatoid arthritis.

As used interchangeably herein, the terms “methionine aminopeptidase 2 inhibitor” and “MetAP-2 inhibitor” are intended to include any compound which inhibits the activity of the methionine aminopeptidase 2 protein, the well known enzyme which cleaves the N-terminal methionine residue of newly synthesized proteins to produce the active form of the protein. In a preferred embodiment, MetAP-2 inhibitors useful in the methods of the invention include those inhibitors comprising a Fumagillin core, such as the ones described in sub-section I below.

As used herein, the term “subject” includes warm-blooded animals, preferably mammals, including humans. In a preferred embodiment, the subject is a primate. In an even more preferred embodiment, the subject is a human.

As used herein, the term “administering” to a subject includes dispensing, delivering or applying a MetAP-2 inhibitor compound, e.g., a MetAP-2 inhibitor in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

As used herein, the term “effective amount” includes an amount effective, at dosages and for periods of time necessary, to achieve the desired result, e.g., sufficient to treat a bone associated disease in a subject. An effective amount of a MetAP-2 inhibitor, as defined herein, may vary according to factors such as the disease state, age, and weight of the subject, and the ability of the MetAP-2 inhibitor to elicit a desired response in the subject. Dosage regimens may be adjusted to provide the optimum therapeutic response. An effective amount is also one in which any toxic or detrimental effects (e.g., side effects) of the MetAP-2 inhibitor are outweighed by the therapeutically beneficial effects.

A therapeutically effective amount of a compound of the invention (i.e., an effective dosage) may range from about 0.001 to 30 mg/kg body weight, preferably about 0.01 to 25 mg/kg body weight, more preferably about 0.1 to 30 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including, but not limited to, the severity of the disease or disorder, previous treatments, the general health and/or age of the subject, and other diseases present, if any. Moreover, treatment of a subject with a therapeutically effective amount of a compound of the invention can include a single treatment or, preferably, can include a series of treatments. In one example, a subject is treated with a compound of the invention in the range of between about 0.1 and 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound used for treatment may increase or decrease over the course of a particular treatment.

I. Methionine Aminopeptidase 2 (MetAP-2) Inhibitors

Any methionine aminopeptidase 2 (MetAP-2) inhibitor capable of inhibiting the activity of the methionine aminopeptidase 2 protein may be used in the methods of the present invention. Such inhibitors are well known in the art and include those described in, for example, U.S. Pat. No. 6,548,477 B1; U.S. Pat. No. 6,919,307; U.S. Publication No. US-2005-0239878-A1; U.S. Pat. No. 5,135,919; U.S. Pat. No. 5,180,738; U.S. Pat. No. 5,290,807; U.S. Pat. No. 5,648,382; U.S. Pat. No. 5,698,586; U.S. Pat. No. 5,767,293; U.S. Pat. No. 5,789,405, the contents of each of which are incorporated herein by reference.

In a preferred embodiment, the MetAP-2 inhibitor is a compound of Formula I, 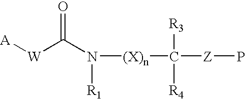
In Formula I, A is a MetAP-2 inhibitory core, W is O or NR₂, and R₁ and R₂ are each, independently, hydrogen or alkyl; X is alkylene or substituted alkylene, preferably linear C₁-C₆-alkylene; n is 0 or 1; R₃ and R₄ are each, independently, hydrogen, substituted or unsubstituted alkyl, substituted or unsubstituted aryl or arylalkyl or substituted or unsubstituted heteroaryl or heteroalkyl. R₃ and R₄ can also, together with the carbon atom to which they are attached, form a carbocyclic or heterocyclic group; or R₁ and R₄ together can form an alkylene group; Z is —C(O)—, alkylene-C(O)— or alkylene; and P is a peptide comprising from 1 to about 100 amino acid residues attached at its amino terminus to Z or a group OR₅ or N(R₆)R₇, wherein R₅, R₆ and R₇ are each, independently, hydrogen, alkyl, substituted alkyl, azacycloalkyl or substituted azacycloalkyl. R₆ and R₇ can also form, together with the nitrogen atom to which they are attached, a substituted or unsubstituted heterocyclic ring structure.

In another embodiment of the compounds of Formula I, W, X, n, R₁, R₃ and R₄ have the meanings given above for these variables; Z is —O—, —NR₈—, alkylene-O— or alkylene-NR₈—, where R₈ is hydrogen or alkyl; and P is hydrogen, alkyl, preferably normal or branched C₁-C₄-alkyl or a peptide consisting of from 1 to about 100 amino acid residues attached at its carboxy terminus to Z.

In compounds of Formula I, when any of R₁-R₈ is an alkyl group, preferred alkyl groups are substituted or unsubstituted normal, branched or cyclic C₁-C₆ alkyl groups. Particularly preferred alkyl groups are normal or branched C₁-C₄ alkyl groups. A substituted alkyl group includes at least one non-hydrogen substituent, such as an amino group, an alkylamino group or a dialkylamino group; a halogen, such as a fluoro, chloro, bromo or iodo substituent; or hydroxyl.

When at least one of R₃ and R₄ is a substituted or unsubstituted aryl or heteroaryl group, preferred groups include substituted and unsubstituted phenyl, naphthyl, indolyl, imidazolyl and pyridyl. When at least one of R₃ and R₄ is substituted or unsubstituted arylalkyl or heteroarylalkyl, preferred groups include substituted and unsubstituted benzyl, naphthylmethyl, indolylmethyl, imidazolylmethyl and pyridylmethyl groups. Preferred substituents on aryl, heteroaryl, arylalkyl and heteroarylalkyl groups are independently selected from the group consisting of amino, alkyl-substituted amino, halogens, such as fluoro, chloro, bromo and iodo; hydroxyl groups and alkyl groups, preferably normal or branched C₁-C₆-alkyl groups, most preferably methyl groups. X is preferably linear C₁-C₆-alkylene, more preferably C₁-C₄-alkylene and most preferably methylene or ethylene. When Z is alkylene-C(O)—, alkylene-O— or alkylene-NR₈, the alkylene group is preferably linear C₁-C₆-alkylene, more preferably C₁-C₄-alkylene and most preferably methylene or ethylene.

R₆ and R₇, in addition to alkyl, substituted alkyl or hydrogen, can each also independently be a substituted or unsubstituted azacycloalkyl group or a substituted or unsubstituted azacycloalkylalkyl group. Suitable substituted azacycloalkyl groups include azacycloalkyl groups which have an N-alkyl substituent, preferably an N—C₁-C₄-alkyl substituent and more preferably an N-methyl substituent. R₆ and R₇ can also, together with the nitrogen atom to which they are attached, form a heterocyclic ring system, such as a substituted or unsubstituted five or six-membered aza- or diazacycloalkyl group. Preferably, the diazacycloalkyl group includes an N-alkyl substituent, such as an N—C₁-C₄-alkyl substituent or, more preferably, an N-methyl substituent.

In particularly preferred embodiments, —N(R₆)R₇ is NH₂ or one of the groups shown below:

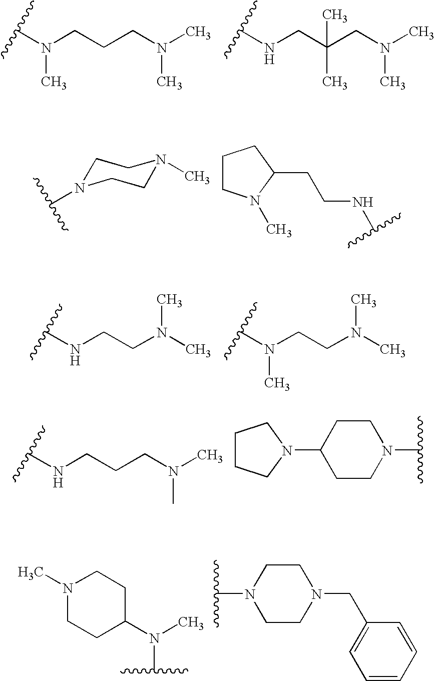

Preferably, the compounds of Formula I do not include compounds wherein Z is —O—, P is hydrogen, R₃ and R₄ are both hydrogen, n is 1 and X is methylene. Preferably, the compounds of Formula I further do not include compounds wherein Z is methylene-O—, R₃ and R₄ are both hydrogen, and n is 0.

In another embodiment, the MetAP-2 inhibitor is a compound of Formula XV, 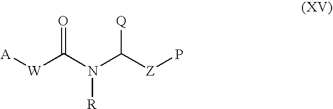
where A is a MetAP-2 inhibitory core and W is O or NR. In one embodiment, Z is —C(O)— or -alkylene-C(O)— and P is NHR, OR or a peptide consisting of one to about one hundred amino acid residues connected at the N-terminus to Z. In this embodiment, Q is hydrogen, linear, branched or cyclic alkyl or aryl, provided that when P is —OR, Q is not hydrogen. Z is preferably —C(O)— or C₁-C₄-alkylene-C(O)—, and, more preferably, —C(O)— or C₁-C₂-alkylene-C(O)—. Q is preferably linear, branched or cyclic C₁-C₆-alkyl, phenyl or naphthyl. More preferably, Q is isopropyl, phenyl or cyclohexyl.

In another embodiment, Z is -alkylene-O— or -alkylene-N(R)—, where alkylene is, preferably, C₁-C₆-alkylene, more preferably C₁-C₄-alkylene and, most preferably, C₁-C₂-alkylene. P is hydrogen or a peptide consisting of from one to about one hundred amino acid residues connected to Z at the carboxyl terminus. In this embodiment, Q is hydrogen, linear, branched or cyclic alkyl or aryl, provided that when P is hydrogen, Q is not hydrogen. Q is preferably linear, branched or cyclic C₁-C₆-alkyl , phenyl or naphthyl. More preferably, Q is isopropyl, phenyl or cyclohexyl.

In the compounds of Formula XV, each R is, independently, hydrogen or alkyl. In one embodiment, each R is, independently, hydrogen or linear, branched or cyclic C₁-C₆-alkyl. Preferably, each R is, independently, hydrogen or linear or branched C₁-C₄-alkyl. More preferably, each R is, independently, hydrogen or methyl. In the most preferred embodiments, each R is hydrogen.

In Formulas I and XV, A is a MetAP-2 inhibitory core. As used herein, a “MetAP-2 inhibitory core” includes a moiety able to inhibit the activity of methionine aminopeptidase 2 (MetAP-2), e.g., the ability of MetAP-2 to cleave the N-terminal methionine residue of newly synthesized proteins to produce the active form of the protein. Preferred MetAP-2 inhibitory cores are Fumagillin derived structures.

Suitable MetAP-2 inhibitory cores include the cores of Formula II, 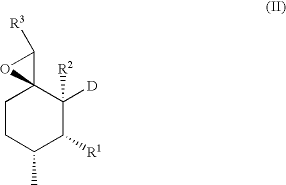
where R¹ is hydrogen or alkoxy, preferably C₁-C₄-alkoxy and more preferably, methoxy. R² is hydrogen or hydroxy; and R³ is hydrogen or alkyl, preferably C₁-C₄-alkyl and more preferably, hydrogen. D is linear or branched alkyl, preferably C₁-C₆-alkyl; arylalkyl, preferably aryl-C₁-C₄-alkyl and more preferably phenyl-C₁-C₄-alkyl; or D is of the structure 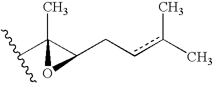
where the dashed line represents a single bond or a double bond.

“A” can also be a MetAP-2 inhibitory core of Formula III, 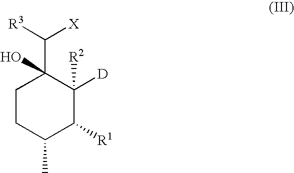
Where R¹, R², R³ and D have the meanings given above for Formula II, and X is a leaving group, such as a halogen.

Examples of suitable MetAP-2 inhibitory cores include, but are not limited to, the following.

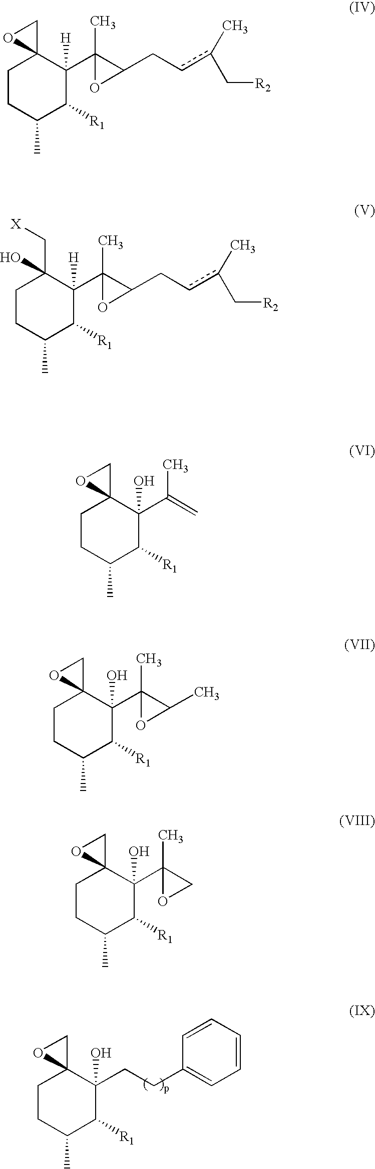

In each of Formulas IV-X, the indicated valence on the ring carbon is the point of attachment of the structural variable W, as set forth in Formulas I-XV. In Formula IX, p is an integer from 0 to 10, preferably 1-4. In Formulas IV, V and VI-IX, R₁ is hydrogen or C₁-C₄-alkoxy, preferably methoxy. In Formulas IV and V, the dashed line indicates that the bond can be a double bond or a single bond. In Formula V, X represents a leaving group, such as a thioalkoxy group, a thioaryloxy group, a halogen or a dialkylsulfinium group. In Formulas IV and V, R₂ is H, OH, amino, C₁-C₄-alkylamino or di(C₁-C₄-alkyl)amino), preferably H. In formulas in which the stereochemistry of a particular stereocenter is not indicated, that stereocenter can have either of the possible stereochemistries, consistent with the ability of the MetAP-2 inhibitor to inhibit the activity of MetAP-2.

In particularly preferred embodiments, A is the MetAP-2 inhibitory core of Formula X below.

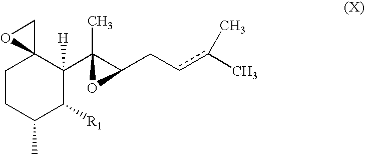

As used herein, the terms “P” and “peptide” include compounds comprising from 1 to about 100 amino acid residues (e.g., 1, 2, 3, 4, 5, 6, 7, 8, 9, 10, 11, 12, 13, 14, 15, 16, 17, 18, 19, 20 or more amino acid residues). In preferred embodiments, the peptide includes compounds comprising less than about 90, 80, 70, 60, 50, 40, 30, 20, or amino acid residues, preferably about 1-10, 1-20, 1-30, 1-40, 1-50, 1-60, 1-70, 1-80, or 1-90 amino acid residues. The peptides may be natural or synthetically made. The amino acid residues are preferably α-amino acid residues. For example, the amino acid residues can be independently selected from among the twenty naturally occurring amino acid residues, the D-enantiomers of the twenty natural amino acid residues, and may also be non-natural amino acid residues (e.g., norleucine, norvaline, phenylglycine, β-alanine, or a peptide mimetic such as 3-amino-methylbenzoic acid). In one embodiment, the amino acid residues are independently selected from residues of Formula XI, Formula XII, and Formula XIII.

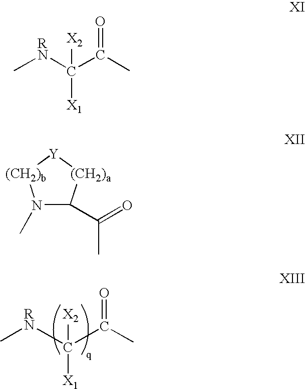

In Formula XI, X₁ is hydrogen, a side chain of one of the twenty naturally-occurring amino acid residues, a linear, branched or cyclic C₁-C₈-alkyl group, an aryl group, such as a phenyl or naphthyl group, an aryl-C₁-C₄-alkyl group, a heteroaryl group, such as a pyridyl, thienyl, pyrrolyl, or furyl group, or a heteroaryl-C₁-C₄-alkyl group; and X₂ is hydrogen a linear, branched or cyclic C₁-C₈-alkyl group, an aryl group, such as a phenyl or naphthyl group, an aryl-C₁-C₄-alkyl group or a heteroaryl group as described above for X₁. Preferably, X₂ is hydrogen. In Formula XII, Y is methylene, oxygen, sulfur or NH, and a and b are each, independently, 0-4, provided that the sum of a and b is between 1 and 4. Formulas XI and XII encompass α-amino acid residues having either a D or an L stereochemistry at the alpha carbon atom. One or more of the amino acid residues can also be an amino acid residue other than an α-amino acid residue, such as a β-, γ- or ε-amino acid residue. Suitable examples of such amino acid residues are of Formula XIII, wherein q is an integer of from 2 to about 6, and each X₁ and X₂ independently have the meanings given above for these variables in Formula XI.

In a preferred embodiment, the peptide used in the MetAP-2 inhibitors used in the methods of the invention may include a site-directed sequence in order to increase the specificity of binding of the MetAP-2 inhibitor to a cell surface of interest. As used herein, the term “site-directed sequence” is intended to include any amino acid sequence (e.g., comprised of natural or non natural amino acid residues) which serves to limit exposure of the MetAP-2 inhibitor to the periphery and/or which serves to direct the MetAP-2 inhibitor to a site of interest, e.g., a site of bone loss.

The peptide contained within the MetAP-2 inhibitors used in the methods of the invention may include a peptide cleavage site for an enzyme which is expressed at sites of bone loss or formation, allowing tissue-selective delivery of a cell-permeable active MetAP-2 inhibitor or fragment thereof (e.g., a fragment containing the MetAP-2 inhibitory core of the MetAP-2 inhibitor). The peptide may also include a sequence which is a ligand for a cell surface receptor which is expressed at a site of bone loss or formation, thereby targeting MetAP-2 inhibitors to a cell surface of interest. However, the selection of a peptide sequence must be such that the active MetAP-2 inhibitor is available to be delivered to the cells in which MetAP-2 inhibition is desired.

The peptide can be attached to the MetAP-2 inhibitory core at either its N-terminus or C-terminus. When the peptide is attached to the MetAP-2 inhibitory core at its C-terminus, the N-terminus of the peptide can be —NR₂R₃, where R₂ is hydrogen, alkyl or arylalkyl and R₃ is hydrogen, alkyl, arylalkyl or acyl. When the peptide is attached to the MetAP-2 inhibitory core at its N-terminus, the C-terminus can be —C(O)R₄, where R₄ is —OH, —O-alkyl, —O-arylalkyl, or —NR₂R₃, where R₂ is hydrogen, alkyl or arylalkyl and R₃ is hydrogen, alkyl, arylalkyl or acyl. In this embodiment, the C-terminal residue can also be present in a reduced form, such as the corresponding primary alcohol.

The methods of the present invention may also utilize pharmaceutically acceptable salts of the MetAP-2 inhibitors described herein. A “pharmaceutically acceptable salt” includes a salt that retains the desired biological activity of the parent MetAP-2 inhibitor and does not impart any undesired toxicological effects. Examples of such salts are salts of acids such as hydrochloric acid, hydrobromic acid, sulfuric acid, phosphoric acid, nitric acid, and the like; acetic acid, oxalic acid, tartaric acid, succinic acid, malic acid, benzoic acid, pamoic acid, alginic acid, methanesulfonic acid, naphthalenesulfonic acid, and the like. Also included are salts of cations such as sodium, potassium, lithium, zinc, copper, barium, bismuth, calcium, and the like; or organic cations such as trialkylammonium. Combinations of the above salts are also useful.

Preferred MetAP-2 Inhibitors of Formula I

One set of particularly preferred MetAP-2 inhibitors to be used in the methods of the invention includes compounds in which A is the MetAP-2 inhibitory core of Formula X, W is O or NR₂, and the structure 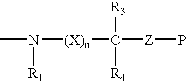
is represented by the structures set forth below.

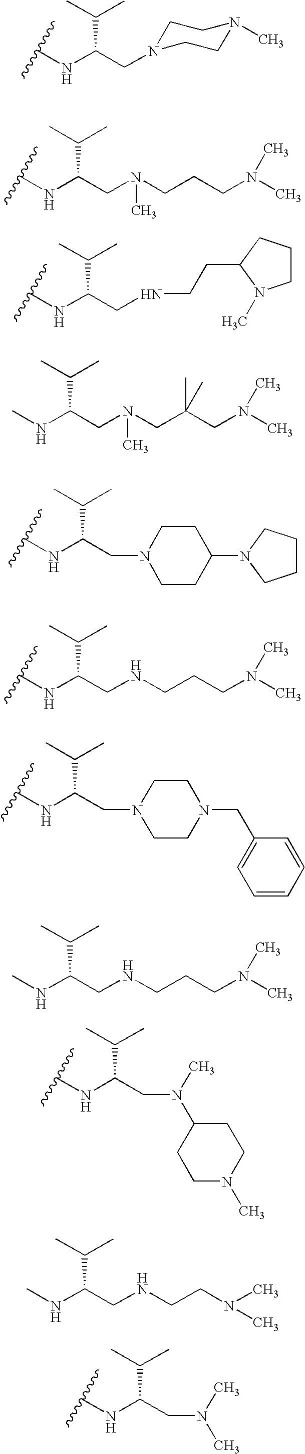

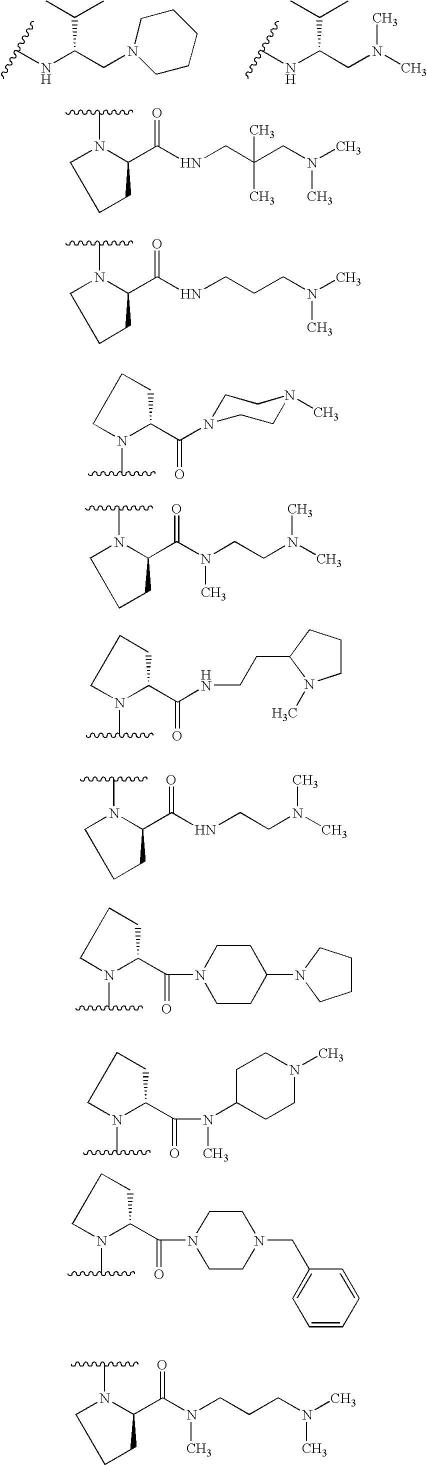

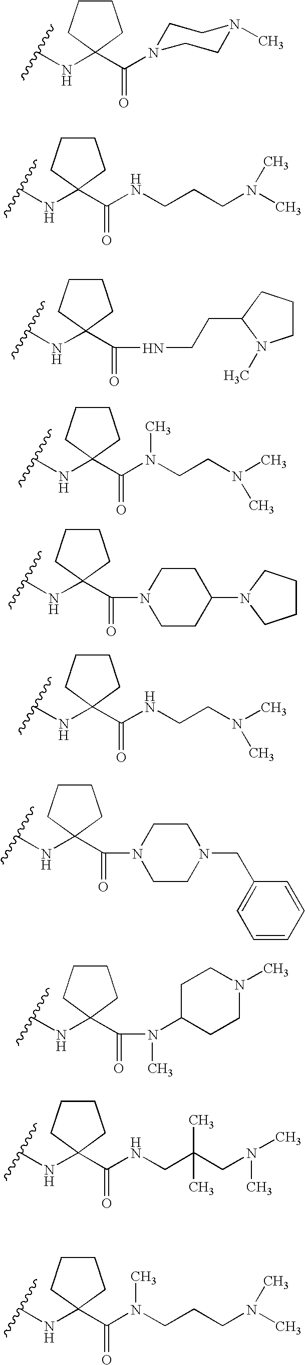

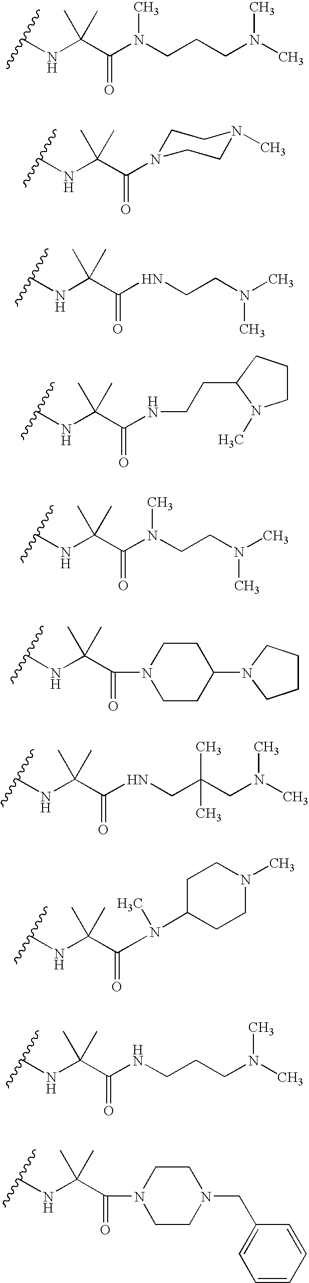

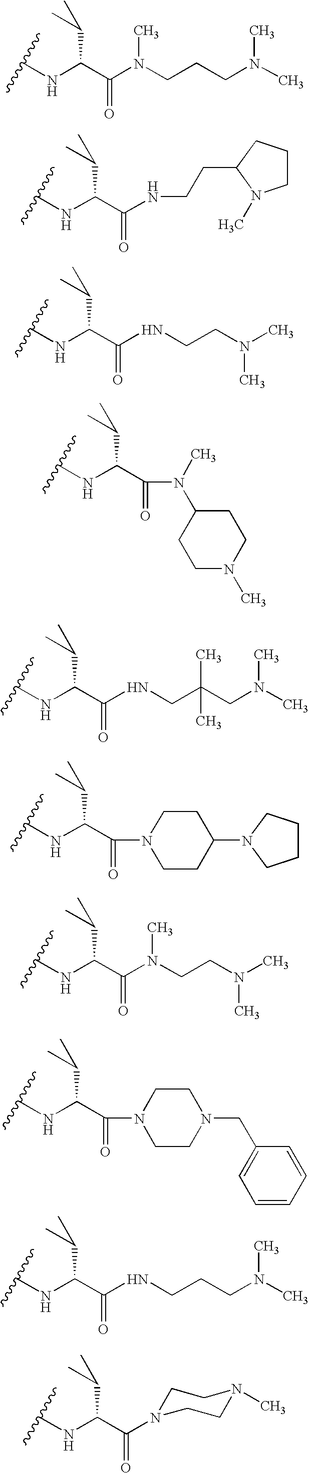

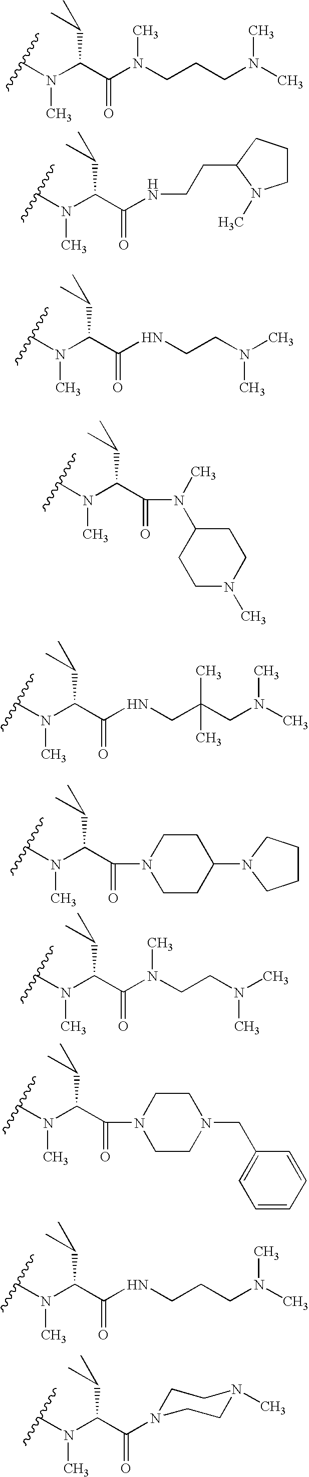

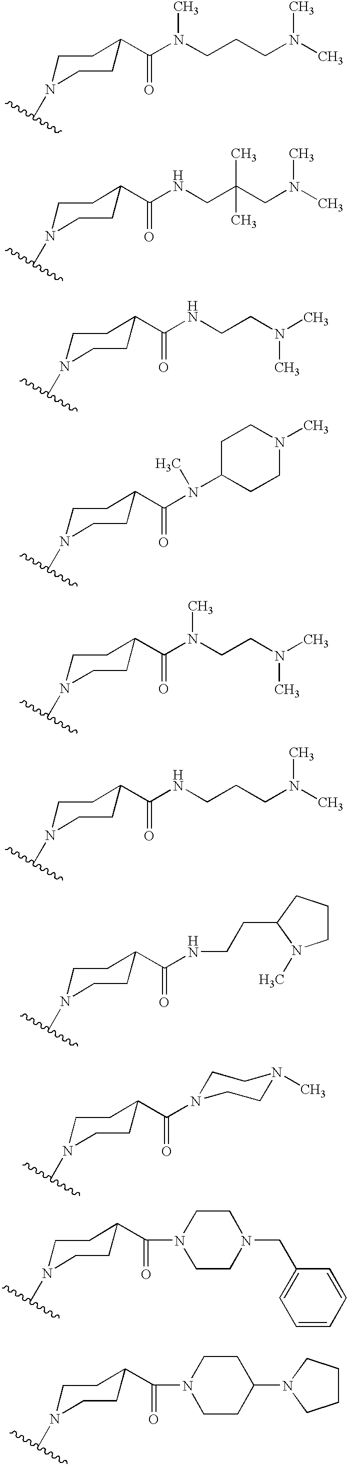

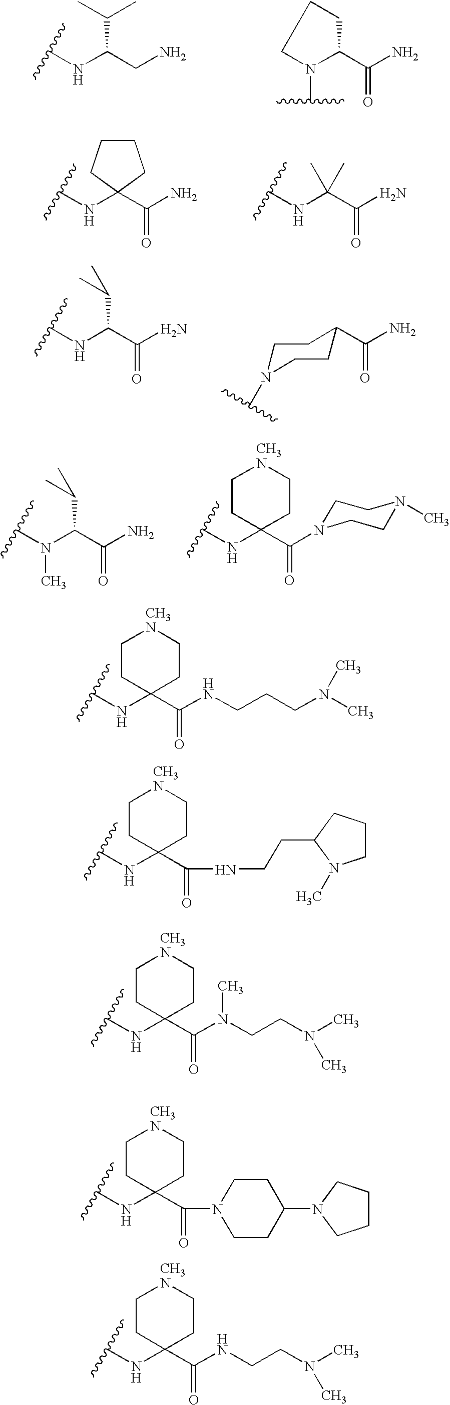

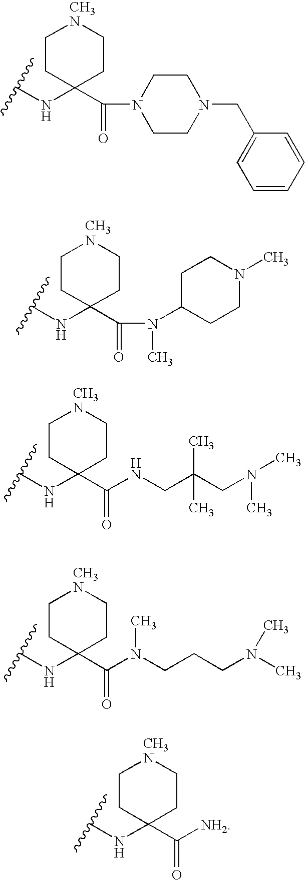

Preferred MetAP-2 Inhibitors of Formula XV

A preferred subset of the MetAP-2 inhibitors of Formula XV to be used in the methods of the invention comprises Formula XIV shown below.

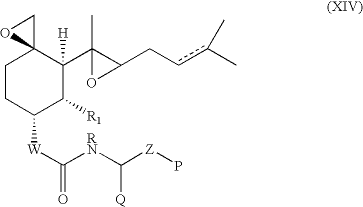

In one embodiment, W is O or NR. Z is —C(±) or -alkylene-C(O)—, preferably C1-C4-alkylene-C(O)—. R is hydrogen or a C₁-C₄-alkyl. Q is hydrogen; linear, branched or cyclic C₁-C₆-alkyl; or aryl. R¹ is hydroxy, C₁-C₄-alkoxy or halogen. P is NH₂, OR or a peptide attached to Z at its N-terminus and comprising from 1 to 100 amino acid residues independently selected from naturally occurring amino acid residues, D-enantiomers of the naturally occurring amino acid residues and non-natural amino acid residues. When Q is H, P is not NH₂ or OR. In preferred embodiments, W is O or NH; Q is isopropyl; R₁ is methoxy; P comprises from 1 to 15 amino acid residues; and the dashed line present in Formula XIV represents a double bond. In particularly preferred embodiments, W is O, and P comprises 10 or fewer amino acid residues.

In another embodiment of the compounds of Formula XIV, W is O or NR. Z is alkylene-O or alkylene-NR, preferably C₁-C₄-alkylene-O or C₁-C₄-alkylene-NR—. R is hydrogen or a C₁-C₄-alkyl. Q is hydrogen; linear, branched or cyclic C₁-C₆-alkyl; or aryl. R₁ is hydroxy, C₁-C₄-alkoxy or halogen. P is hydrogen or a peptide attached to Z at its C-terminus and comprising from 1 to 100 amino acid residues independently selected from naturally occurring amino acid residues, D-enantiomers of the naturally occurring amino acid residues and non-natural amino acid residues. When Q is H, P is not H. In preferred embodiments, W is O or NH; Q is isopropyl; R₁ is methoxy; P comprises from 1 to 15 amino acid residues; and the dashed line present in Formula XIV represents a double bond. In particularly preferred embodiments, W is O, and P comprises 10 or fewer amino acid residues or P is hydrogen.

One set of particularly preferred MetAP-2 inhibitors for use in the methods of the invention is represented by the structures set forth below. 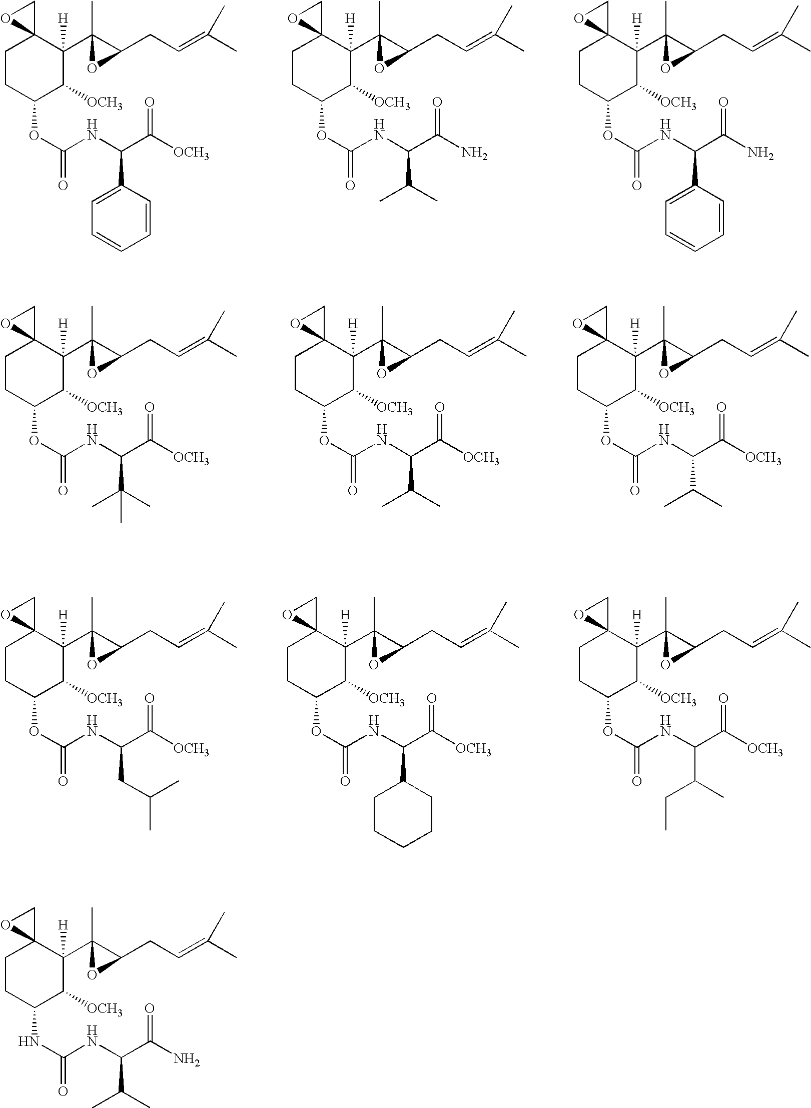 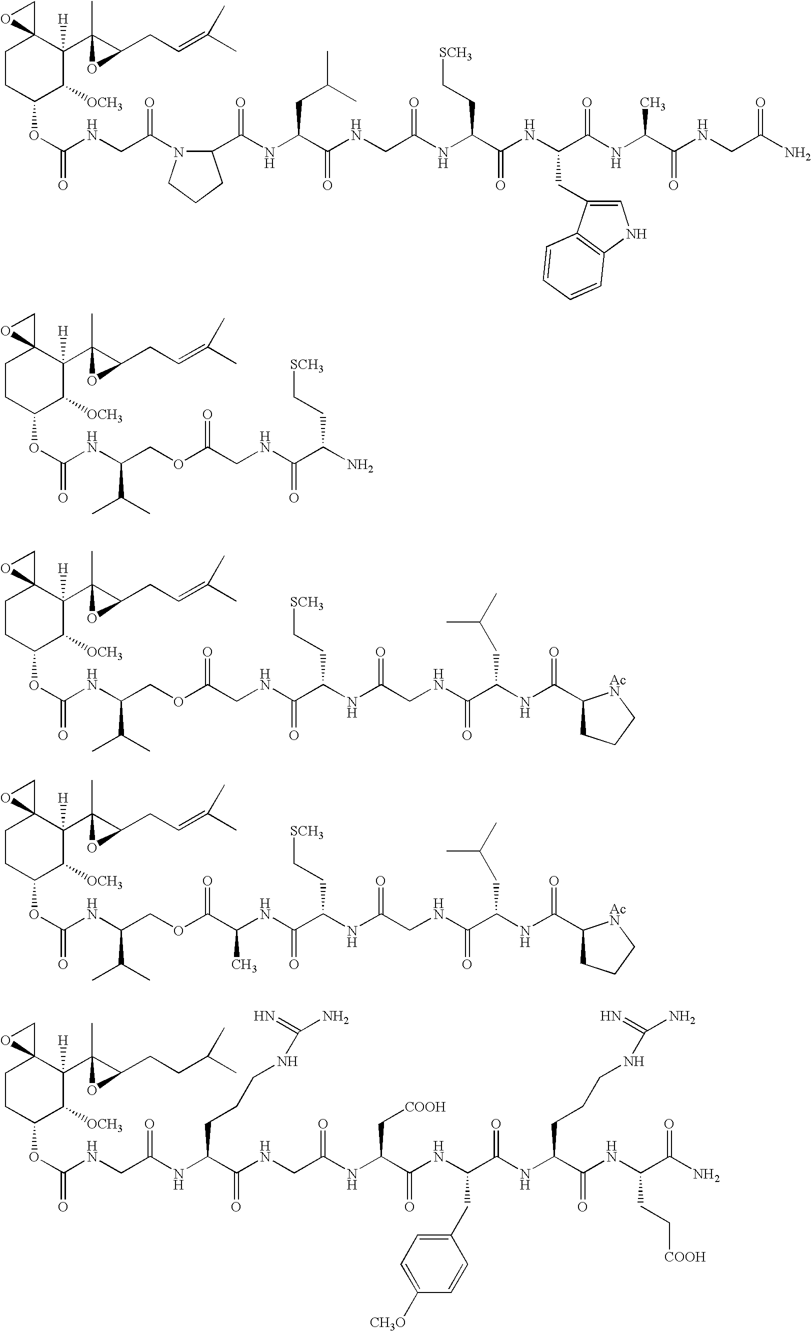 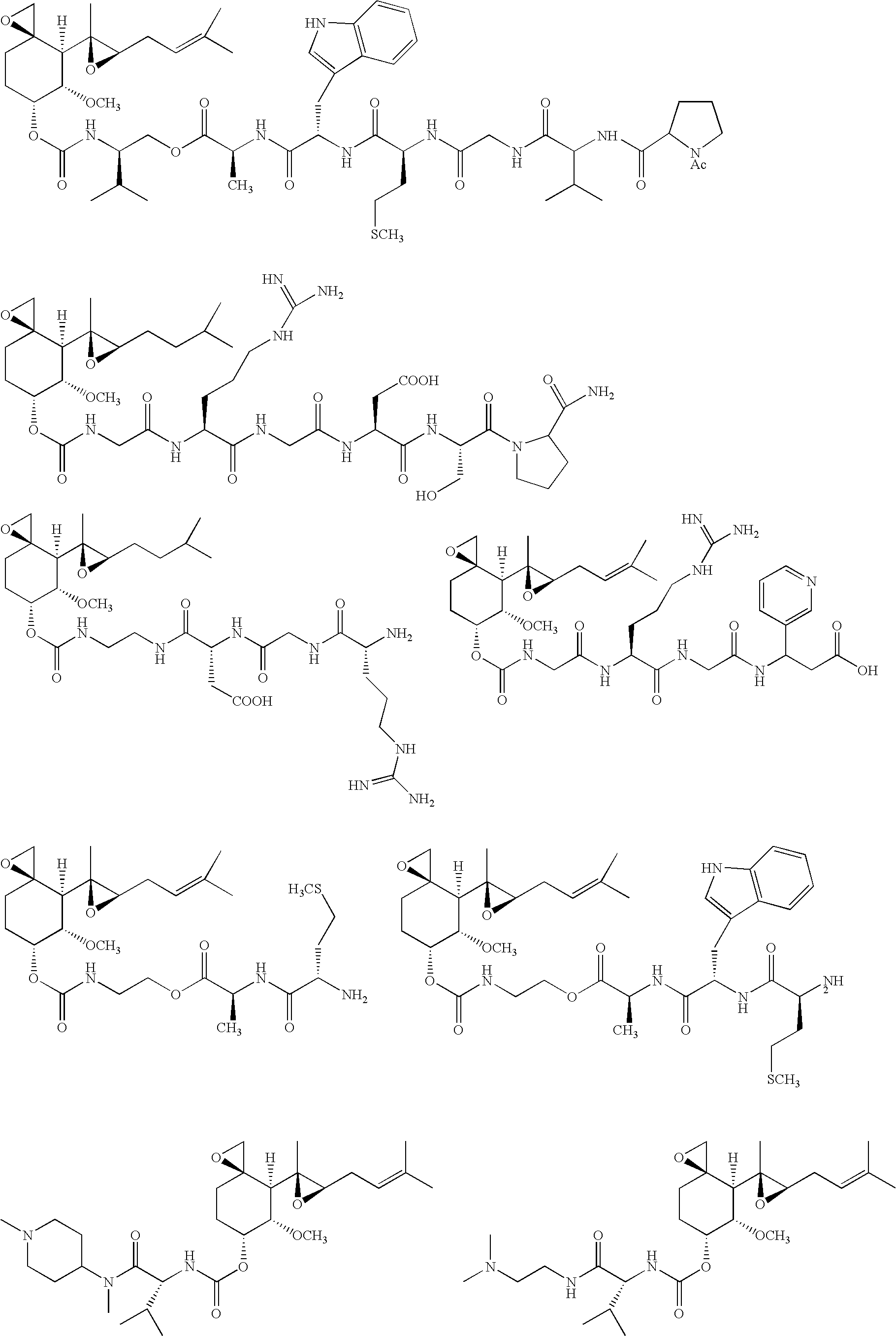 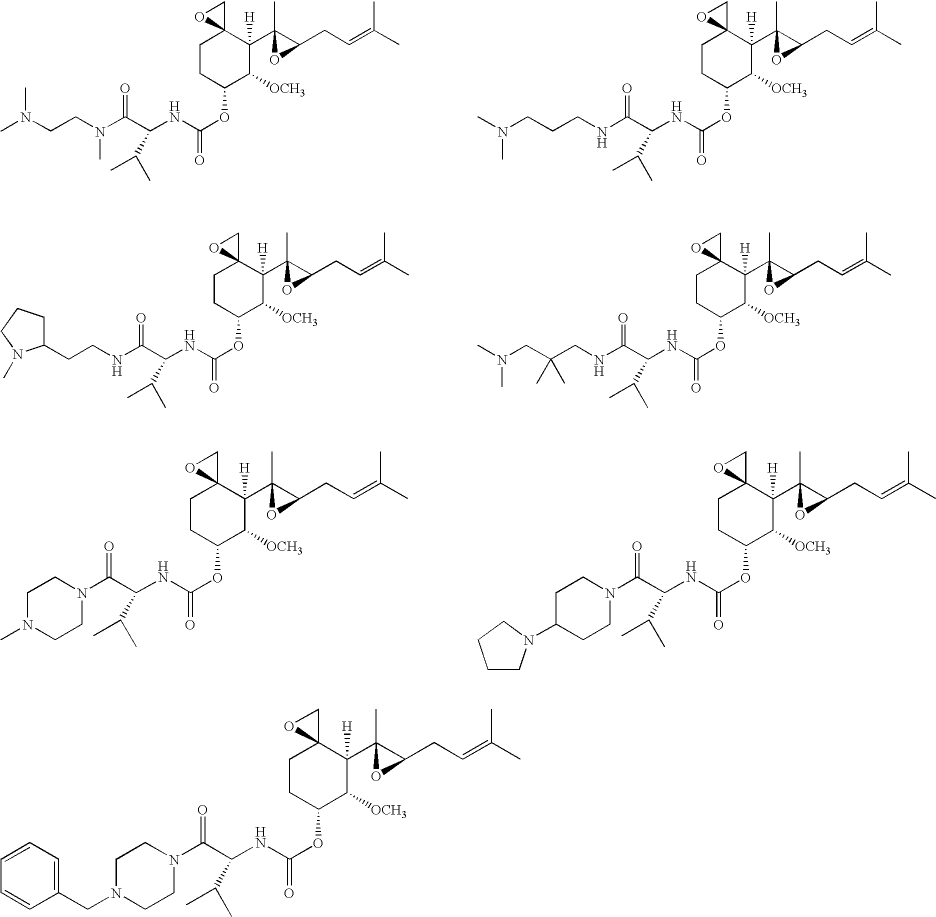
II. Methods of Treatment of Bone Associated Disease

The present invention provides a method of treating a bone associated disease in a subject. The method includes administering to the subject a therapeutically effective amount of a MetAP-2 inhibitor, thereby treating a bone associated disease in the subject.

As used herein, the term “bone associated disease” is intended to include any disease, disorder or condition which the amount of bone in a subject is decreased and/or the structural integrity of the bone is impaired. This bone erosion may be mediated by bone resorption by osteoclasts. Bone associated diseases include, but are not limited to: rheumatoid arthritis, osteoporosis, Paget's Disease, Gorham's Disease, multiple myeloma, bone metastasis of cancer, periodontal disease, renal osteodystrophy, Hajdu-Cheney Syndrome (acro-osteolysis), Idiopathic Multicentric Osteolysis, Multicentric Osteolysis with nephropathy, Torg Osteolysis Syndrome (multicentric osteolysis), Neurogenic osteolysis, Joseph and Shinz Disease (Idiopathic Phalangeal Acro-osteolysis), Winchester Syndrome, Lupus, and Kummell's Disease.

As used herein, the term “subject” includes warm-blooded animals, preferably mammals, including humans. In a preferred embodiment, the subject is a primate. In an even more preferred embodiment, the subject is a human.

As used herein, the term “administering” to a subject includes dispensing, delivering or applying an MetAP-2 inhibitor, e.g., an MetAP-2 inhibitor in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

As used herein, the term “effective amount” includes an amount effective, at dosages and for periods of time necessary, to achieve the desired result, e.g., sufficient to treat a bone associated disease in a subject. An effective amount of a MetAP-2 inhibitor, as defined herein may vary according to factors such as the disease state, age, and weight of the subject, and the ability of the MetAP-2 inhibitor to elicit a desired response in the subject. Dosage regimens may be adjusted to provide the optimum therapeutic response. An effective amount is also one in which any toxic or detrimental effects (e.g., side effects) of the MetAP-2 inhibitor are outweighed by the therapeutically beneficial effects.

A therapeutically effective amount of a compound of the invention (i.e., an effective dosage) may range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including, but not limited to, the severity of the disease or disorder, previous treatments, the general health and/or age of the subject, and other diseases present, if any. Moreover, treatment of a subject with a therapeutically effective amount of a compound of the invention can include a single treatment or, preferably, can include a series of treatments. In one example, a subject is treated with a compound of the invention in the range of between about 0.1 and 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound used for treatment may increase or decrease over the course of a particular treatment.

The methods of the invention further include administering to a subject a therapeutically effective amount of a MetAP-2 inhibitor in combination with another pharmaceutically active compound known to treat a bone associated disease. Supplementary pharmaceutically active compounds known to treat bone associated diseases, including non-steroidal anti-inflammatory agents (NSAIDs), e.g., diclofenac, diflunisal, etodolac, flurbiprofen, ibuprofen, indomethacin, ketoprofen, ketorolac, nabumetone, naproxen, oxaprozin, piroxicam, sulindac, tolmetin; cortocosteroids, e.g., predinose, predinsolone, decadron (dexamethasone), triamcinolone, and deflazacort; disease modifying anti-rheumatic drugs (DMARDs), e.g., methotrexate, hydroxychloroquine, sulfasalazine, leflunomide, TNF-inhibitors, solid IL-1 receptor therapy, intramuscular gold, azathioprine, cyclophosphamide, and cyclosporine A; selective estrogen receptor modulators (SERMs), e.g., raloxifene; bisphosphonates, e.g., alendronate, risedronate, etidronate, calcitonin, and sodium monofluorophosphate; or compounds that may potentiate the ability of the MetAP-2 inhibitor to inhibit osteoclast differentiation can also be incorporated into the compositions of the invention. Suitable pharmaceutically active compounds that may be used can be found in Harrison's Principles of Internal Medicine, Thirteenth Edition, Eds. T. R. Harrison et al. McGraw-Hill: N.Y., NY; and the Physicians Desk Reference 50th Edition 1997, Oradell, New Jersey, Medical Economics Co., the complete contents of which are expressly incorporated herein by reference. The compound of the invention and the other pharmaceutically active compound may be administered to the subject in the same pharmaceutical composition or in different pharmaceutical compositions (at the same time or at different times).

III. Pharmaceutical Compositions

The MetAP-2 inhibitors to be used in the methods of the present invention are preferably administered to a subject using a pharmaceutically acceptable formulation. Such pharmaceutically acceptable formulations typically include one or more MetAP-2 inhibitors as well as a pharmaceutically acceptable carrier(s) and/or excipient(s). As used herein, “pharmaceutically acceptable carrier” includes any and all solvents, dispersion media, coatings, antibacterial and antifungal agents, isotonic and absorption delaying agents, and the like that are physiologically compatible. The use of such media and agents for pharmaceutically active substances is well known in the art. Except insofar as any conventional media or agent is incompatible with the compounds of the invention, use thereof in the pharmaceutical compositions is contemplated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection include sterile aqueous solutions (where water soluble), or dispersions and sterile powders for the extemporaneous preparation of sterile solutions or dispersions for injection. For intravenous administration, suitable carriers include physiological saline, bacteriostatic water, Cremophor EL™ (BASF, Parsippany, N.J.) or phosphate buffered saline (PBS). In all cases, the pharmaceutical composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols, such as mannitol or sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the compound of the invention in the required amount in an appropriate solvent with one or a combination of the ingredients enumerated above, as required, followed by filtered sterilization. Generally, dispersions are prepared by incorporating the MetAP-2 inhibitor into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the MetAP-2 inhibitor plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the MetAP-2 inhibitor can be incorporated with excipients and used in the form of tablets, troches, or capsules. Oral compositions can also include an enteric coating. Oral compositions can also be prepared using a fluid carrier for use as a mouthwash, wherein the MetAP-2 inhibitor in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the MetAP-2 inhibitors are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the MetAP-2 inhibitors are formulated into ointments, salves, gels, or creams as generally known in the art.

The pharmaceutical compositions of the invention can also be prepared in the form of suppositories (e.g., with conventional suppository bases such as cocoa butter and other glycerides) or retention enemas for rectal delivery.

In one embodiment, the MetAP-2 inhibitors are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811, U.S. Pat. No. 5,455,044, U.S. Pat. No. 5,576,018 and U.S. Pat. No. 4,883,666, the contents of all of which are incorporated herein by reference.

The MetAP-2 inhibitors can also be incorporated into pharmaceutical compositions which allow for the sustained delivery of the MetAP-2 inhibitors to a subject for a period of at least several weeks to a month or more. Such formulations are described in U.S. Pat. No. 5,968,895; U.S. Pat. No. 6,699,833 B1; U.S. Pat. No. 6,180,608 B1; U.S. Publication No. US 2002-0176841 A1; U.S. Publication No. US 2005-0112087 A1; U.S. Publication No. US 2002-0086829 A1, the contents of each of which are incorporated herein by reference.

It is especially advantageous to formulate oral or parenteral compositions in unit dosage form for ease of administration and uniformity of dosage. Unit dosage form, as used herein, refers to physically discrete units suited as unitary dosages for the subject to be treated; each unit containing a predetermined quantity of one or more compounds of the invention calculated to produce the desired therapeutic effect in association with the required pharmaceutical carrier. The specification for the unit dosage forms of the invention are dictated by and directly dependent on the unique characteristics of the therapeutic compound and the particular therapeutic effect to be achieved, and the limitations inherent in the art of compounding such compounds for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. MetAP-2 inhibitors which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of the compounds of the invention lies preferably within a range of circulating concentrations that include the ED50 with little or no toxicity. The dosage may vary within this range depending upon the dosage form employed and the route of administration utilized. For any compounds used in the methods of the invention, the therapeutically effective dose can be estimated initially from cell culture assays. A dose may be formulated in animal models to achieve a circulating plasma concentration range that includes the IC50 (i.e., the concentration of the compound which achieves a half-maximal inhibition of symptoms) as determined in cell culture.

Such information can be used to more accurately determine useful doses in humans.

Levels in plasma may be measured, for example, by high performance liquid chromatography.

This invention is further illustrated by the following examples which should not be construed as limiting. The contents of all references, patents and published patent applications cited throughout this application, as well as the Figures are hereby incorporated by reference.

### EXAMPLES

Materials and Methods for Examples 1-5

Reagents. The MetAP-2 inhibitor comprising the structure (1-Carbamoyl-2-methyl-propyl)-carbamic acid-(3R,4S,5S,6R)-5-methoxy-4-[(2R,3R)-2-methyl-3-(3-methyl-but-2-enyl)-oxiranyl]-1-oxa-spiro[2,5]oct-6-yl ester was used in these experiments. For in vitro studies, a 10 mM stock solution in ethanol was prepared. For in vivo administration, the MetAP-2 inhibitor was dissolved in 11% 2-hydroxypropyl-beta-cyclodextran (HPCD) (Cargill Incorporation). PG-PS was obtained from Lee Biomolecular Laboratories, dexamethasone (4 mg/ml) from Henry Schein, and E-64 from Sigma.

Osteoclast differentiation assays. Primary human osteoclast (OC) precursors (Cambrex) were seeded at 10,000 cells/well (50,000 cells/ml) in osteoclast precursor growth medium (Cambrex). The cells were cultured for 7 days in the presence of either M-CSF (33 ng/ml), M-CSF (33 ng/ml) and RANKL (33 ng/ml), or in the presence of both cytokines and different concentrations of the MetAP-2 inhibitor. OC differentiation was determined by staining for the OC marker TRAP, using a leukocyte acid phosphatase kit (Sigma). Briefly, after 7 days in culture, the cells were rinsed once with PBS, fixed with 37% formaldehyde in acetone-citrate buffer for 1 min, and stained for development of red color according to the manufacturer's instructions.

Rat model of PG-PS induced arthritis. Female Lewis rats (109-130 g) were received from Charles River Laboratories. Food and water were available ad libitum. PG-PS (25 mg/kg) was injected i.p. on day 1 and responding animals were randomized into treatment groups on day 14. Vehicle (11% HPCD in PBS), dex and the MetAP-2 inhibitor (1, 5, and 10 mg/kg) were administered p.o., qod. Paw swelling was monitored using a plethysmometer (Stoelting Co., Woodale, Ill.) according to instrument specifications. The volumes of the two hind paws were measured and averaged on day 1, 4, 6, 8, 10, 13, 15, 17, 20, 22, 23, 27, 29 and 31. Ten animals were assigned to each group except the vehicle group and animals which received no PG-PS, but 10 mg/kg MetAP-2 inhibitor (n=4).

Clinical assessment of PG-PS arthritis. The histopathological evaluation was performed on the left and right hind joints of randomly selected animals from each study group by an independent histopathologist without knowledge of specific interventions. After completion of the study, the left and right hind ankles were removed, fixed in 10% buffered formalin, decalcified in 5% formic acid, paraffin-embedded, sectioned, and H&E stained for histological evaluation. A joint histology scoring system which grades the severity of 4 histopathological processes (cell infiltration, pannus formation, cartilage erosion, bone resorption), was used to quantify hind joint involvement in the PG-PS arthritis model (36). Dependent on the assigned score for each parameter (0=normal, 1=minimal, 2=mild, 3=moderate, 4=marked), the maximum total score per ankle is 16, and 32 per animal.

MetAP-2 pharmacodynamic assay. The MetAP-2 assay measures the amount of uninhibited MetAP-2 in cells or tissues which has not been derivitized by prior treatment with the MetAP-2 inhibitor (Bernier (2004) Proc. Natl. Acad. Sci. USA 101: 10768-10773 and Bernier (2005) J. Cell. Biochem. 95: 1191-1203). Briefly, wbc from animals of each study group were pooled and cell lysates were prepared as previously described (Bernier (2004) Proc. Natl. Acad. Sci. USA 101: 10768-10773 and Bernier (2005) J. Cell. Biochem. 95: 1191-1203). 10 μg to 20 μg of wbc protein was incubated with a biotinylated analog of the MetAP-2 inhibitor which covalently binds to the catalytic site of MetAP-2. The biotinylated MetAP-2-inhibitor complex was captured on a plate with immobilized streptavidin (Pierce), and detected with the MetAP-2 antibody CM33 (0.5 μg/ml), followed by horseradish peroxidase-conjugated goat anti-rabbit IgG secondary antibody. The amount of uninhibited MetAP-2 was determined by measuring the absorption at 450 nm using a Labsystems Multiskan plate spectrophotometer. Human recombinant MetAP-2 (Mediomics), pre-bound to the biotinylated MetAP-2 inhibitor, was used to generate the standard curve. The detection limit of this assay was 0.47 ng MetAP-2 protein/mg wbc protein.

ELISAs for cartilage and bone biochemical turnover markers. The amount of COMP in serum was measured with a competitive enzyme immunoassay (MD Biosciences, Inc) according to the manufacturer's instructions. The detection limit of this ELISA was 0.2 U/L. Helical peptide (amino acids 620 to 633) from the α1 chain of bone-specific human CTX-I was measured either in cell culture supernatants of primary human OC precursors cultured as described above, or in urine with a competitive enzyme immunoassay (Quidel Corporation). The detection limit of this ELISA was 8 μg/L. All CTX-I measurements in urine were corrected for urinary creatinine excretion for each sample to account for potential differences in renal clearance rates among the different study groups. Urinary creatinine was measured with a colorimetric assay (Quidel Corporation).

Microfocal computed tomography. All specimens were scanned on a Scanco Medical AG μCT 40 system. Images were obtained with an isotropic voxel resolution of 20 microns. A matrix size of 1024×1024 with 1000 projections was utilized for all scans. A total of 1836 slices were scanned for each specimen (the number of slices scanned was determined by the length of the scan needed to cover the entire ankle including the distal tibia). The scan time per specimen was approximately 2.6 hours. The images were then volume rendered using a fixed threshold (at two different thresholds: 255 and 140). The total bone volume and BMD were calculated over the same regions of all the specimens. Micro-CT was performed at Scanco USA, Incorporated.

### Example 1

### The MetAP-2 Inhibitor Inhibits OC Differentiation and Bone Resorption In Vitro

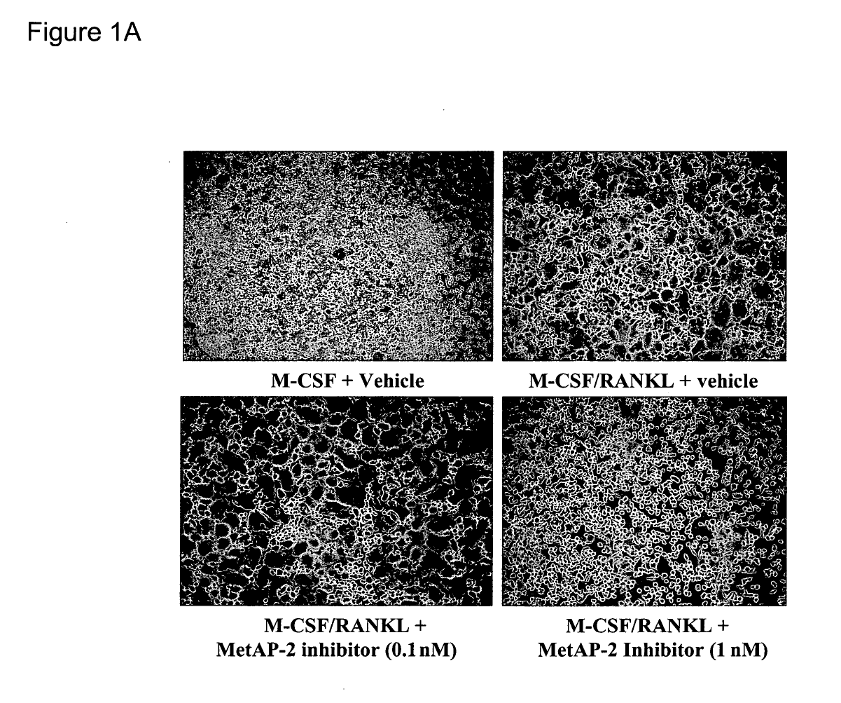

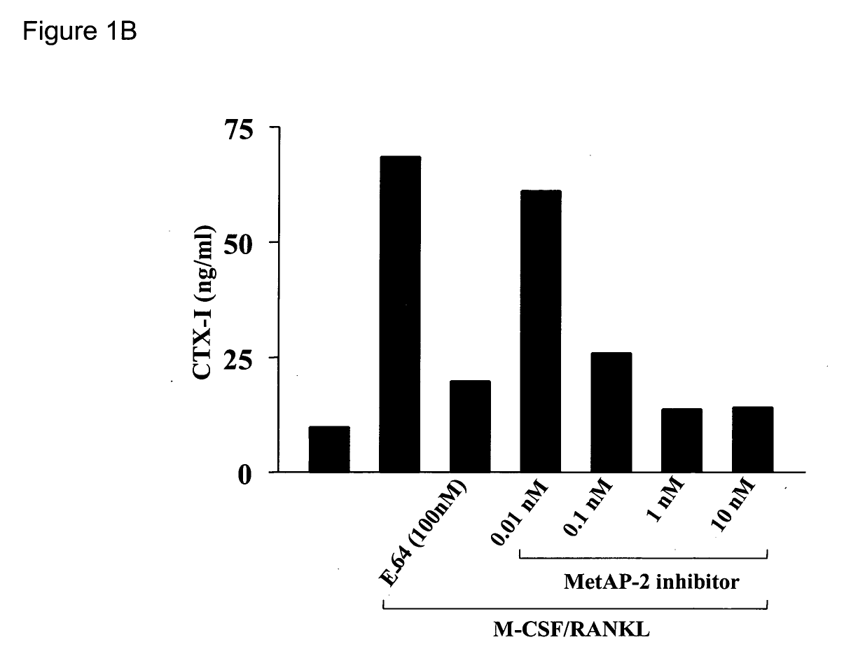

The MetAP-2 inhibitor used in the present studies is an orally available, irreversible MetAP-2 inhibitor of the fumagillin class of molecules that has previously been shown to potently inhibit the proliferation of HUVEC and HFLS-R^{A} in vitro, both cell types which are known for their critical roles in the bone associated disease, rheumatoid arthritis (RA) (Bernier (2004) Proc. Natl. Acad. Sci. USA 101: 10768-10773 and Bernier (2005) J. Cell. Biochem. 95: 1191-1203). The instant invention features such an inhibitor. In order to investigate the activity of the MetAP-2 inhibitor on osteoclast (OC) differentiation and bone resorption in vitro, yet another cell type critically associated with R^{A} pathogenesis in an in vitro osteoclastogenesis model was utilized. Primary human OC precursors were cultured for 7 days in the presence of M-CSF and RANKL, and vehicle or increasing concentrations of the MetAP-2 inhibitor. Cells cultured with M-CSF and RANKL differentiated into large, multinucleated OC, as demonstrated by the appearance of numerous tartrate resistant acid phosphatase (TRAP) stained cells, while the MetAP-2 inhibitor at concentrations at ≦1 nM almost completely inhibited the generation of TRAP-positive mononuclear and multinucleated OC (FIG. 1A). The ability of the MetAP-2 inhibitor to inhibit OC differentiation was fully reversible. Furthermore, the incubation of these cells with the MetAP-2 inhibitor at concentrations up to 100 nM did not induce cytotoxicity, consistent with previous results that the exposure of HUVEC and HFLS-R^{A} to the MetAP-2 inhibitor (100 nM) did not induce apoptosis (Bernier (2004) Proc. Natl. Acad. Sci. USA 101: 10768-10773).

To determine whether the inhibition of OC differentiation by the MetAP-2 inhibitor in vitro correlated with the inhibition of bone resorption in vitro, primary human OC were cultured on a thin layer of human bone particles in the presence of M-CSF and RANKL, and vehicle or increasing concentrations of the MetAP-2 inhibitor. The non-specific cysteine proteinase inhibitor E-64 (100 nM), a known inhibitor of bone resorption in vitro, was used as a control. After 7 days, the culture supernatant was collected and the amount of bone-specific collagen type I C-terminal helical peptide (CTX-I) was measured by ELISA. The MetAP-2 inhibitor potently inhibited the bone resorbing activity of OC in a dose-dependent manner (IC₅₀≦0.1 nM), and the degree of inhibition at 1 nM and 10 nM was comparable to the inhibitory activity of E-64 at 100 nM (FIG. 1B). Notably, this marked inhibition of bone resorption occurred at a concentration (≦0.1 nM) that showed no detectable inhibition of OC differentiation by this agent.

### Example 2

### Potent Anti-Inflammatory Activity of the MetAP-2 Inhibitor in a Rat Arthritis Model is Correlated with the Inhibition of MetAP-2 Function

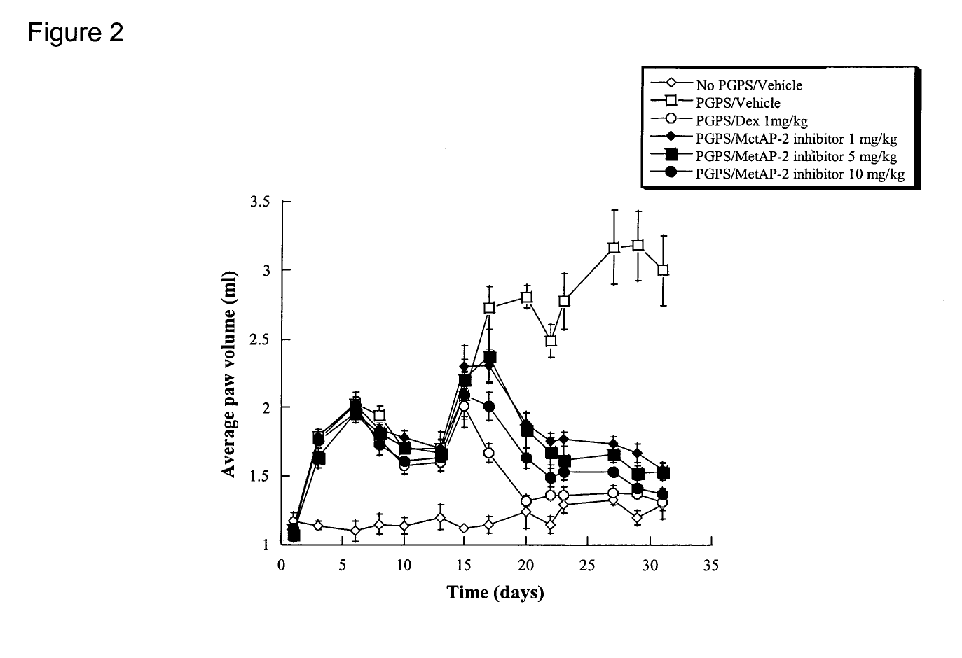

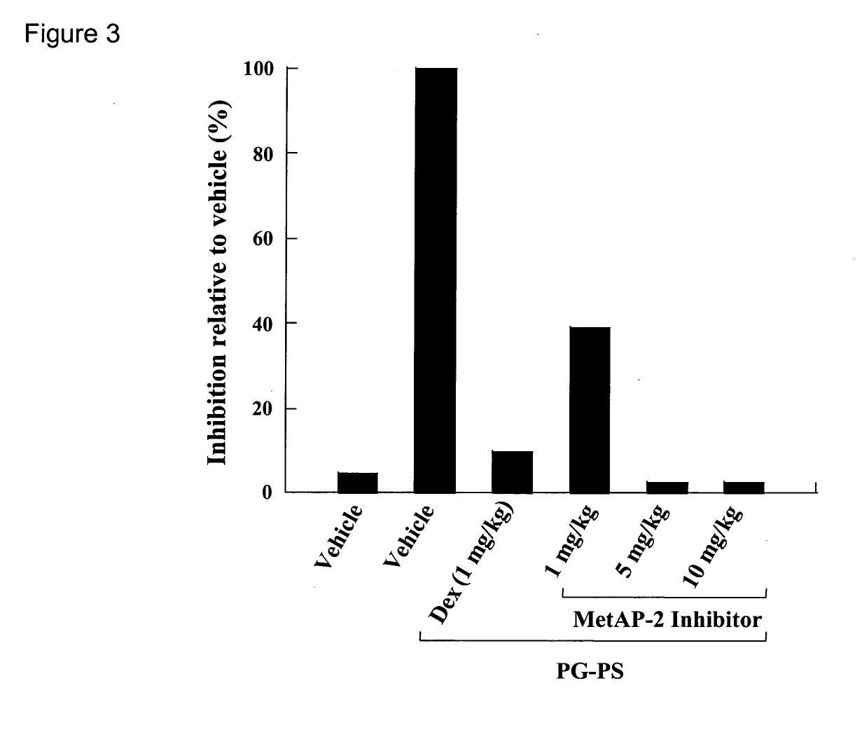

Since the MetAP-2 inhibitor had the ability to inhibit multiple cell types critical for pathogenesis of the bone associated disease, RA, in vitro, it was hypothesized that the observations from the in vitro studies (Example 1) would translate into protection from disease in animals in the PG-PS model of arthritis. The progression of disease in this model follows a biphasic mode, with an early acute, predominantly neutrophil-driven phase which persists to days 6-7, followed by a chronic, T cell dependent phase (evident around day 12), which is characterized by chronic inflammation and erosive synovitis (Palombella (1998) Proc. Natl. Acad. Sci. USA 95:15671-15676). Therapeutic dosing of animals administered the MetAP-2 inhibitor orally (p.o.) at 1, 5 and 10 mg/kg, every other day (qod), or vehicle started at day 15 after the chronic destructive phase of the disease was established and terminated on day 31. Consistent with previous results, the MetAP-2 inhibitor at all 3 doses demonstrated significant amelioration of joint swelling and inflammation, measured by paw swelling of the hind limbs, when compared to vehicle-treated animals (FIG. 2) (Bernier (2004) Proc. Natl. Acad. Sci. USA 101: 10768-10773). It was next examined whether the protective activity of the MetAP-2 inhibitor in this model was linked to the inhibition of the molecular target MetAP-2, similar to the previously observed growth inhibition of HUVEC and HFLS-RA in vitro, which was directly correlated with the amount of MetAP-2 inhibited (Bernier (2004) Proc. Natl. Acad. Sci. USA 101: 10768-10773). The amount of uninhibited MetAP-2 in wbc of animals from all treatment groups was measured after conclusion of the study, using the MetAP-2 pharmacodynamic assay (Bernier (2004) Proc. Natl. Acad. Sci. USA 101: 10768-10773 and Bernier (2005) J. Cell. Biochem. 95: 1191-1203). In animals orally administered the MetAP-2 inhibitor at 1, 5 and 10 mg/kg, qod, ≧60% of MetAP-2 in wbc was inhibited at the lowest dose, while ≧95% of MetAP-2 was inhibited at 5 and 10 mg/kg, relative to the vehicle-treated group (FIG. 3). These results demonstrated that the protective activity of the MetAP-2 inhibitor observed in vivo was linked to the inhibition of MetAP-2 function, and confirmed that the amount of uninhibited MetAP-2 in wbc could serve as a pharmacodynamic marker to measure the activity of the MetAP-2 inhibitor in an experimental model of arthritis. Notably, ≧90% MetAP-2 inhibition was also observed after the administration of dexamethasone (dex) (1 mg/kg, p.o., qod). No MetAP-2 inhibition was observed in naïve animals treated with dex for 12 days at 1 mg/kg, qod, every 4 days or every 6 days, suggesting a potentially novel mechanism of protection from disease for steroids in experimental arthritis.

### Example 3

### Protection from Experimental Arthritis by the MetAP-2 Inhibitor is Provided Through Suppression of the Severity of Clinical Indices of Inflammation and Destruction

It was investigated whether the protective activity of the MetAP-2 inhibitor in this animal model of arthritis, which is characterized by aggressive synovitis, extensive pannus formation, cartilage degradation and focal bone erosion, was mediated through regression in the severity of all clinical indices tested, or whether this activity was targeting specific pathogenic processes. Therapeutic dosing of the MetAP-2 inhibitor (1, 5, 10 mg/kg, p.o., qod) significantly decreased the total arthritic score and extensive protection ranging from 50% to 80% was observed for all clinical indices, compared to vehicle-treated animals (Table 1), with the highest level of protection observed for inhibition of cartilage erosion at a dose of 10 mg/kg. These results demonstrated that the protection of animals from arthritis in this model was mediated through a significant decrease in all clinical indices of inflammatory and destructive processes.

### Example 4

### Structural Damage in Affected Joints Through Cartilage Erosion and Bone Destruction is Significantly Inhibited by the MetAP-2 Inhibitor

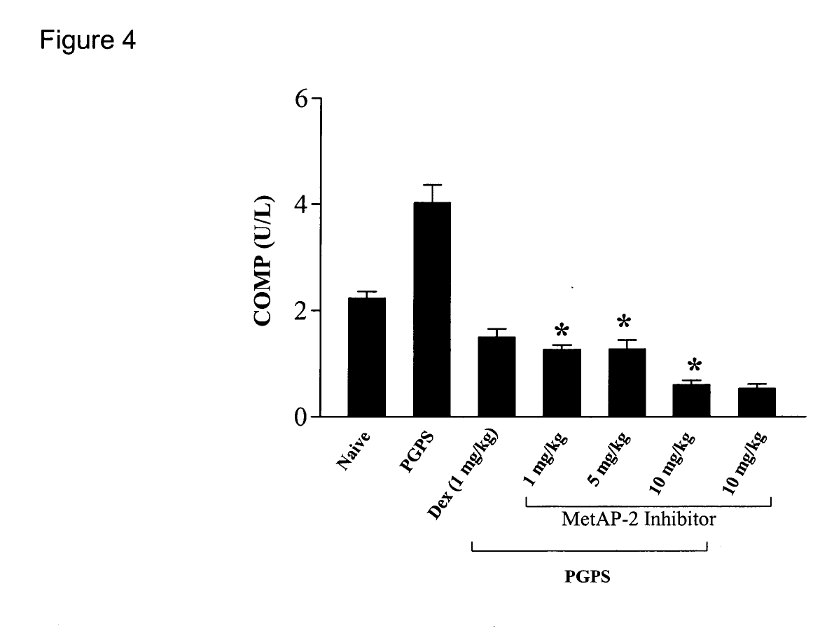

The destruction of articular joints is the radiographic hallmark of bone associated disease. Therefore, the activity of the MetAP-2 inhibitor on these destructive processes was assessed by measuring biochemical turnover markers of cartilage erosion and bone resorption. COMP is a major component of the extracellular matrix of the muscoskeletal system that mediates chondrocyte attachment through interactions with integrins (Chen (2005) J. Biol. Chem. 280:32655-32661). The amount of COMP in serum of animals treated therapeutically with the MetAP-2 inhibitor (1, 5, and 10 mg/kg, p.o., qod), or vehicle was measured after conclusion of the study. A significant decrease in systemic levels of this marker, even below the level of serum COMP measured in naïve animals treated with vehicle, was detected after treatment with all doses of the MetAP-2 inhibitor (FIG. 4), consistent with the clinical assessment for cartilage erosion (Table 1).

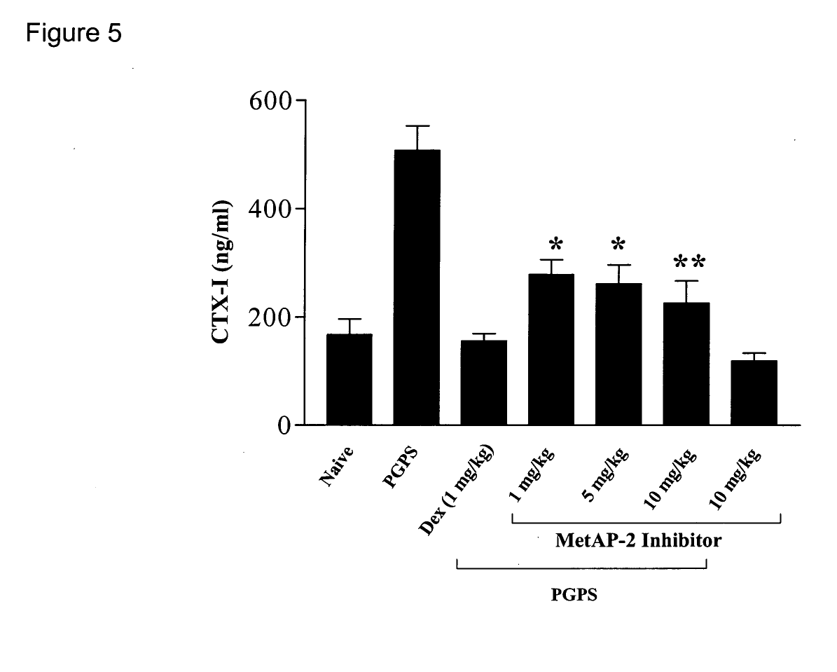

To assess the activity of the MetAP-2 inhibitor on systemic bone resorption in vivo, the amount of systemic CTX-I in urine was measured and corrected for urinary creatinine excretion after conclusion of the study. Therapeutic administration of the MetAP-2 inhibitor (1, 5 and 10 mg/kg, p.o., qod) resulted in significantly decreased systemic urinary CTX-I levels compared to vehicle-treated animals (FIG. 5). These results confirmed that the MetAP-2 inhibitor inhibited bone resorption in this model, consistent with the clinical assessment of this parameter (Table 1).

TABLE 1

| Group | Cell infiltration | Pannus formation | Cartilage erosion | Bone Resorption | Total arthritis score |
|---|---|---|---|---|---|
| Naïve plus vehicle | 0 | 0 | 0 | 0 | 0 |
| PG-PS arthritis plus | 3.45 ± 0.26 | 3.65 ± 0.16 | 3.15 ± 0.19 | 3.60 ± 0.17 | 13.85 ± 0.75 |
| vehicle | | | | | |
| PG-PS arthritis plus | 0.25 ± 0.08^{A} | 0.55 ± 0.08^{A} | 0.60 ± 0.14^{B} | 0.95 ± 0.13^{B} | 2.35 ± 0.23^{B} |
| dex (1 mg/kg) | | | | | |
| PG-PS arthritis plus | 1.85 ± 0.15^{A} | 2.00 ± 0.14^{A} | 1.10 ± 0.12^{B} | 2.05 ± 0.2 | 7.00 ± 0.58^{B} |
| MetAP-2 inhibitor (1 mg/kg) | | | | | |
| PG-PS arthritis plus | 2.15 ± 0.18^{A} | 2.40 ± 0.20^{A} | 1.25 ± 0.18^{B} | 1.80 ± 0.20^{B} | 7.60 ± 0.67^{B} |
| MetAP-2 inhibitor (5 mg/kg) | | | | | |
| PG-PS arthritis plus | 1.38 ± 0.31^{A} | 1.72 ± 0.20^{A} | 0.72 ± 0.14^{B} | 2.00 ± 0.16^{B} | 5.83 ± 0.71^{B} |
| MetAP-2 inhibitor (10 mg/kg) | | | | | |

Table 1 demonstrates that the MetAP-2 inhibitor used in the present studies suppresses the severity of clinical indices of arthritis. A joint histology scoring system which grades the severity of 4 histopathological processes (cell infiltration, pannus formation, cartilage erosion, bone resorption) was used (O'Byrne (1991) Agents and Actions 134:239-241).

### Example 5

### The MetAP-2 Inhibitor Preserves the Structural Integrity of Hind Joints and Prevents the Loss of Bone Volume and BMD

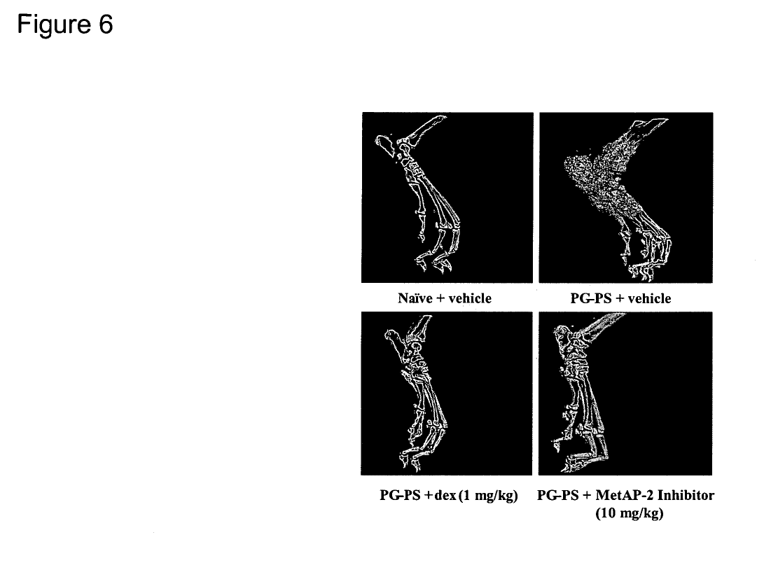

Finally, the activity of the MetAP-2 inhibitor on the structural preservation of hind joints in rats with established disease was investigated. Three-dimensional rendered micro-CT images of representative rat hind paws, which allow for the non-destructive visualization of pathological joint changes, demonstrated that therapeutic dosing of the MetAP-2 inhibitor at 10 mg/kg, p.o., qod, protected the structural integrity of the joints and prevented focal bone erosions, compared to vehicle-treated animals, which showed significant bone erosion and compromised joint integrity (FIG. 6). Moreover, treatment with the MetAP-2 inhibitor preserved bone volume and showed protection from BMD loss, compared to vehicle-treated animals, as determined by quantitative micro-CT analysis (Table 2).

These data demonstrate the potent inhibition in vitro of multiple effector cell types critical to the pathogenesis of RA and other bone associated diseases by the MetAP-2 inhibitor. They also demonstrate the disease-modifying activity of the MetAP-2 inhibitor in a rat model of established chronic disease through a mechanism of molecularly targeted inhibition of MetAP-2, verified by the marked suppression of joint inflammation and joint destruction. These data demonstrate the therapeutic potential of the MetAP-2 inhibitor in treating bone associated diseases.

TABLE 2

| Group | Bone Volume (Vox-BV) | BMD (mg HA/ccm) |
|---|---|---|
| Naïve plus vehicle | 206.7 ± 8.93 | 1161 ± 1.15 |
| PG-PS arthritis plus | 162.6 ± 9.91 | 1044 ± 17.88 |
| vehicle | | |
| PG-PS arthritis plus | 240.7 ± 12.68 | 1125 ± 2.59 |
| dex (1 mg/kg) | | |
| PG-PS arthritis plus | 213.8 ± 5.23 | 1087 ± 17.44 |
| MetAP-2 inhibitor | | |
| (10 mg/kg) | | |

Table 2 shows that the MetAP-2 inhibitor used in the present studies prevents loss of total bone volume and attenuates the loss of BMD. The ankles including the distal tibia were scanned using a Scanco Medical AG μCT 40 system (threshold: 255), and the total bone volume and BMD were calculated over the same regions of all specimens.

### Example 6

### The Effects of MetAP-2 Inhibitor Treatment on an Animal Model for Osteoporosis

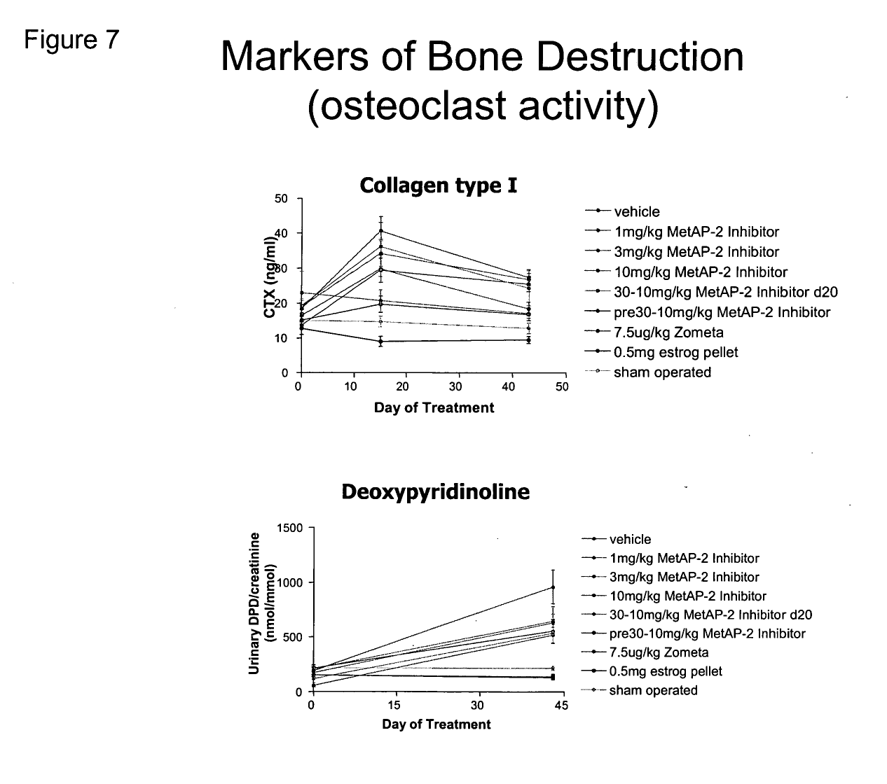

An animal model of osteoporosis (the CD rat described in, for example, Glatt M. et al. (2004) Osteoporos. Int. 15:707-715) was used to determine the effect of treatment with MetAP-2 inhibitors as described herein. Briefly, aged rats were ovarectomised to mimic post-menopausal osteoporosis and the rats were treated with the MetAP-2 inhibitor and various controls as described in FIG. 7. At different times during the treatment, urinary samples were collected from the animals and using an ELIZA assay the amount of: (A) a C-terminal helical polypeptide of Collagen type I or (B) deoxypyridinoline (a further break down product of (A)) was determined. The results from these assays are depicted in FIG. 7.

### EQUIVALENTS

Those skilled in the art will recognize, or be able to ascertain using no more that routine experimentation, many equivalents to the specific embodiments of the invention described herein. Such equivalents are intended to be encompassed by the following claims.

We claim:

1. A method of treating a bone associated disease in a subject, comprising administering to the subject a therapeutically effective amount of a methionine aminopeptidase 2 inhibitor, thereby treating a bone associated disease in a subject.

2. The method of claim 1, wherein said bone associated disease is selected from the group consisting of Paget's Disease, Gorham's Disease, multiple myeloma, bone metastasis of cancer, periodontal disease, renal osteodystrophy, Hajdu-Cheney Syndrome (acro-osteolysis), Idiopathic Multicentric Osteolysis, Multicentric Osteolysis with nephropathy, Torg Osteolysis Syndrome (multicentric osteolysis), Neurogenic osteolysis, Joseph and Shinz Disease (Idiopathic Phalangeal Acro-osteolysis), Winchester Syndrome, Lupus, and Kummell's Disease.

3. The method of claim 1, wherein said bone associated disease is osteoporosis.

4. The method of claim 1, wherein said subject is a mammal.

5. The method of claim 4, wherein said mammal is a human.

6. The method of claim 4, wherein said mammal is a female human.

7. The method of claim 1, wherein said methionine aminopeptidase 2 inhibitor is a compound of Formula I, 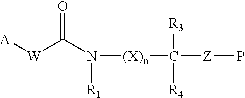
wherein
A is a Met-AP2 inhibitory core;
W is O or NR₂;
R₁ and R₂ are each, independently, hydrogen or alkyl;
X is alkylene or substituted alkylene;
n is 0 or 1;
R₃ and R₄ are each, independently, hydrogen, substituted or unsubstituted alkyl, substituted or unsubstituted aryl or substituted or unsubstituted heteroaryl; or R₃ and R₄, together with the carbon atom to which they are attached, form a carbocyclic or heterocyclic group; or R₃ and R₄ together form an alkylene group;
Z is —C(O)— or alkylene-C(O)—; and
P is a peptide comprising from 1 to about 100 amino acid residues attached at its amino terminus to Z or a group OR₅ or N(R₆)R₇, wherein
R₅, R₆ and R₇ are each, independently, hydrogen, alkyl, substituted alkyl, azacycloalkyl or substituted azacycloalkyl; or R₆ and R₇, together with the nitrogen atom to which they are attached, form a substituted or unsubstituted heterocyclic ring structure;
or
Z is —O—, —NR₈—, alkylene-O— or alkylene-NR₈—, where R₈ is hydrogen or alkyl; and
P is hydrogen, alkyl or a peptide consisting of from 1 to about 100 amino acid residues attached at its carboxy terminus to Z.

8. The method of claim 1, wherein said methionine aminopeptidase 2 inhibitor is a compound of Formula XV, 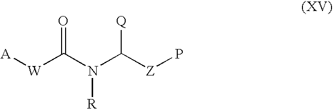
wherein
A is a MetAP-2 inhibitory core;
W is O or NR;
each R is, independently, hydrogen or alkyl;
Z is —C(O)— or -alkylene-C(O)—;
P is NHR, OR or a peptide consisting of one to about one hundred amino acid residues connected at the N-terminus to Z;
Q is hydrogen, linear, branched or cyclic alkyl or aryl, provided that when P is —OR, Q is not hydrogen;
or
Z is -alkylene-O— or -alkylene-N(R)—;
P is hydrogen or a peptide consisting of from one to about one hundred amino acid residues connected to Z at the carboxyl terminus;
Q is hydrogen, linear, branched or cyclic alkyl or aryl, provided that when P is hydrogen, Q is not hydrogen;
and pharmaceutically acceptable salts thereof.

9. The method of claim 1, wherein said methionine aminopeptidase 2 inhibitor is a compound of the formula 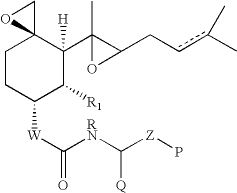
wherein
W is O or NR;
each R is, independently hydrogen or a C₁-C₄-alkyl;
Q is hydrogen; linear, branched or cyclic C₁-C₆-alkyl; or aryl;
R₁ is hydroxy, C₁-C₄-alkoxy or halogen;
Z is —C(O)— or C₁-C₄-alkylene;
P is NHR, OR, or a peptide comprising 1 to 100 amino acid residues attached to Z at the N-terminus; or
Z is alkylene-O or alkylene-NR; and
P is hydrogen or peptide comprising 1 to 100 amino acid residues attached to Z at the C-terminus;
or a pharmaceutically acceptable salt thereof; provided that when P is hydrogen, NHR or OR, Q is not hydrogen.

10. The method of claim 1, wherein said methionine aminopeptidase 2 inhibitor is a compound comprising the structure 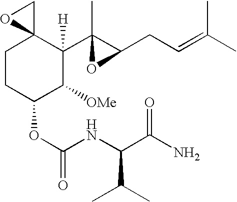
or a pharmaceutically acceptable salt thereof.

11. The method of claim 1, wherein said methionine aminopeptidase 2 inhibitor is a compound comprising the structure (1-Carbamoyl-2-methyl-propyl)-carbamic acid-(3R,4S,5S,6R)-5-methoxy-4-[(2R,3R)-2-methyl-3-(3-methyl-but-2-enyl)-oxiranyl]-1-oxa-spiro[2.5]oct-6-yl ester, or a pharmaceutically acceptable salt thereof.

12. The method of claim 1, wherein said methionine aminopeptidase 2 inhibitor is administered at a dosage range of about 0.1 and 30 mg/kg.

13. The method of claim 1, wherein said methionine aminopeptidase 2 inhibitor is administered at a dosage range of about 0.1 and 10 mg/kg.

14. The method of claim 1, wherein said methionine aminopeptidase 2 inhibitor is administered in a sustained-release formulation.

15. The method of claim 14, wherein said sustained-release formulation provides sustained delivery of the methionine aminopeptidase 2 inhibitor to a subject for at least one week after the formulation is administered to the subject.

16. The method of claim 14, wherein said sustained-release formulation provides sustained delivery of the methionine aminopeptidase 2 inhibitor to a subject for at least two weeks after the formulation is administered to the subject.

17. The method of claim 14, wherein said sustained-release formulation provides sustained delivery of the methionine aminopeptidase 2 inhibitor to a subject for at least three weeks after the formulation is administered to the subject.

18. A method of treating osteoporosis in a subject, comprising administering to the subject a therapeutically effective amount of a methionine aminopeptidase 2 inhibitor comprising the structure (1-Carbamoyl-2-methyl-propyl)-carbamic acid-(3R,4S,5S,6R)-5-methoxy-4-[(2R,3R)-2-methyl-3-(3-methyl-but-2-enyl)-oxiranyl]-1-oxa-spiro[2,5]oct-6-yl ester, or a pharmaceutically acceptable salt thereof, thereby treating osteoporosis in a subject.
